(19) United States
(12) Patent Application Publication
(10) Pub. No.: US 2012/0311744 A1
(43) Pub. Date: Dec. 6, 2012

(54) Marked Cannabis For Indicating Medical Marijuana

(75) Inventor: Erich E. Sirkowski, Dedham, MA (US)

(73) Assignee: Erich E. Sirkowski, Dedham, MA (US)

(21) Appl. No.: 13/490,066

(22) Filed: Jun. 6, 2012

Related U.S. Application Data
(60) Provisional application No. 61/520,115, filed on Jun. 6, 2011.

(51) Int. Cl. A01H 5/00; G01N 21/76; G01N 21/64; C12M 1/34

(52) U.S. Cl. 800/298; 435/288.7; 435/8; 435/18; 250/459.1; 250/458.1; 250/200

(57) ABSTRACT

The invention involves transforming Cannabis with a transgene(s) or chemical(s) expressing biological, chemical, luminescent, and fluorescent markers from the UV, visible, near, mid, and far spectrums of light. This transformation allows for the detection of Medical Marijuana from other forms of marijuana. Cannabis is a genus of flowering plant that include three putative species Cannabis sativa, Cannabis indica, and Cannabis ruderalis. The invention relates to seeds, plants, plant cells, plant tissue, and harvested products from transformed Cannabis. The invention also relates to plants and varieties produced by the method of essential derivation from plants of transformed Cannabis and to plants of transformed Cannabis reproduced by vegetative methods, including but not limited to tissue culture of regenerated cells or tissue from transformed Cannabis.

This application is a non-provisional of U.S. Ser. No. 61/520,115, filed Jun. 6, 2011, entitled “Transformation of Cannabis for Detecting Medical Marijuana.”

This invention relates to methods of integrally marking Cannabis plants, such as for place of production and licensing, plants so marked, and devices for detecting such markings. This invention further relates to the field of plant breeding. More particularly, the invention relates to a variety of Cannabis designated as transformed Cannabis, its essentially derived varieties and the hybrid varieties obtained by crossing transformed Cannabis as a parent line with plants of other varieties or parent lines.

Cannabis is an important, emerging medical option in several states. Due to the importance of Cannabis to the medical and healthcare industry, and to people suffering from debilitating diseases, this invention deals with the important aspect of being able to distinguish Medical Marijuana from common, illegal varieties of Cannabis.

Cannabis is commonly reproduced by self-pollination and fertilization. This type of sexual reproduction facilitates the preservation of plant and variety characteristics during breeding and seed production. The preservation of these characteristics are often important to plant breeders for producing Cannabis plants having desired traits. Other methods of producing Cannabis plants having desired traits are also used and include methods such as genetic transformation via Agrobacterium infection or direct transfer by microparticle bombardment, microinjection, or chemical manipulation. Examples of such methods are disclosed, for example, in U.S. Pub. No. 20090049564, incorporated by reference herein in its entirety.

Due to the environment, the complexity of the structure of genes and location of a gene in the genome, among other factors, it is difficult to engineer the phenotypic expression of a particular genotype. In addition, a plant breeder may only apply his skills on the phenotype and not, or in a very limited way, on the level of the genotype.

By carefully choosing the breeding parents, the breeding and selection methods, the genetic or chemical testing layout and testing locations, the breeder may breed a particular variety type. In addition, a new variety may be tested in special comparative trials (biological or chemical) with other existing varieties in order to determine whether the new variety meets the required characteristics for classification as transformed Cannabis (ie. Medical Marijuana).

What is needed in the art are simple, methods of measuring intrinsic properties in the Cannabis to determine if it is medical marijuana.

### SUMMARY OF THE INVENTION

The invention relates to seeds, plants, plant cells, parts of plants, budding and flowering parts of the transformed Cannabis as well as to hybrid Cannabis plants and seeds obtained by crossing plants of transformed Cannabis with other Cannabis plants. The invention encompasses plants and plant varieties produced by the method of derivation or essential derivation from transformed Cannabis plants and to plants of transformed Cannabis reproduced by vegetative methods, including but not limited to regeneration of embryogenic cells or tissue of transformed Cannabis. The invention also encompasses methods of producing Cannabis seeds that comprise crossing plants of transformed Cannabis either with itself or with a second, distinct Cannabis plant.

An apparatus and/or method for detecting legal Cannabis, and bio-marked Cannabis substantially as shown in and/or described herein, as set forth more completely in the claims.

Various advantages, aspects and novel features of the present disclosure will be more fully understood from the following description and drawings.

### BRIEF DESCRIPTION OF THE DRAWINGS

So that the manner in which the features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1: A detailed illustration of the various components of a Cannabis/marijuana plant. (Cannabis sativa, scientific drawing. from Franz Eugen Köhler's Medizinal-Pflantzen. Published and copyrighted by Gera-Untermhaus, FE Köhler in 1887 (1883-1914). The drawing is signed W. Müller.)

FIG. 2: A diagramatic representation of the Ti-Plasmid from Agrobacterium to be utilized in cloning/engineering of transformed Cannabis for detection of Medical Marijuana from illegal marijuana. This plasmid can be modified to include a bio-marker such as a fluorescent protein of interest gene, and/or a coding-marker for identification of medical marijuana from illegal marijuana.

FIG. 3: An illustration of tobacco transformation to be used to transform Cannabis for expression of fluorescent and/or genetic marker sequences for the detection of Medical Marijuana from illegal marijuana. This diagram shows tobacco being transformed, and the same method can be employed to transform Cannabis to incorporate bio-marker genes and coding-markers for identification of Medical marijuana from illegal marijuana.

FIG. 4: A schematic of a mobile detector of licit Cannabis.

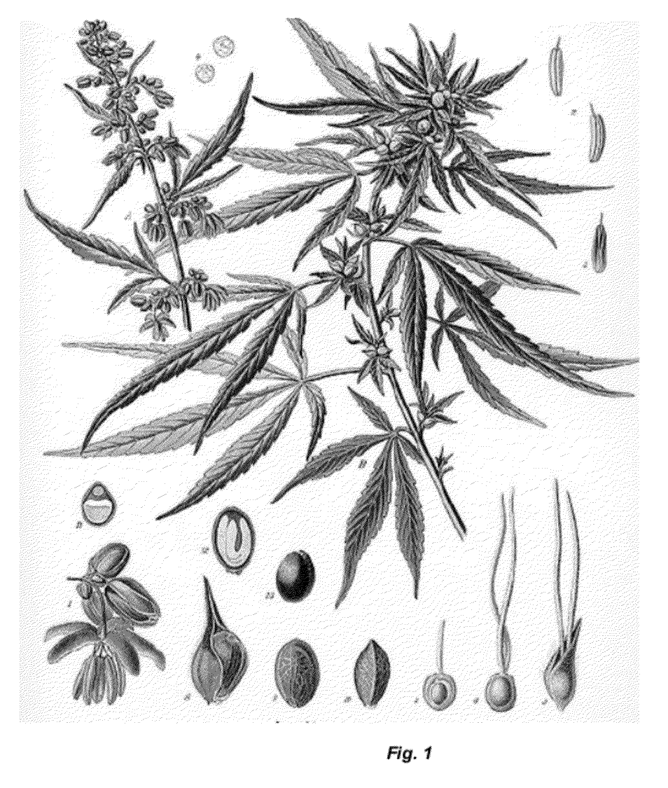

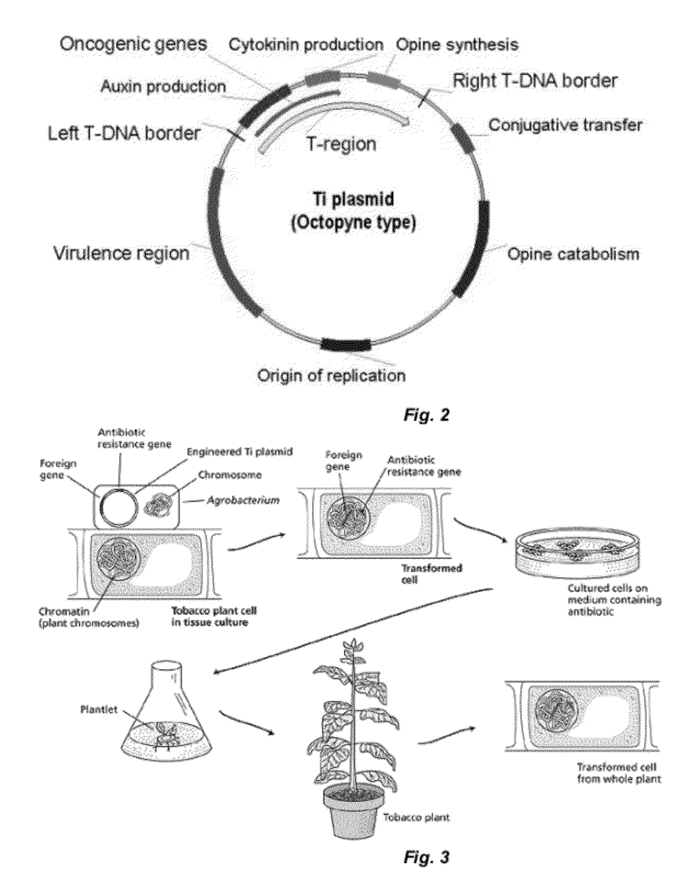

While the invention is described herein by way of example using several embodiments and illustrative drawings, those skilled in the art will recognize that the invention is not limited to the embodiments of drawing or drawings described. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to.

### DETAILED DESCRIPTION

### Scientific Classification

### Kingdom: Plantae

### Division: Magnoliophyta

### Class: Magnoliopsida

### Order: Rosales

### Family: Cannabaceae

### Genus: Cannabis

Species: include without limitation: sativa, indica, ruderalis

The invention can be obtained by physically, chemically and/or biologically transforming Cannabis cells, plants, or tissue, and seeds etc.

Parent plants, which have been selected for good agronomic and fluorescent and genetic quality traits can be manually crossed in different combinations. The resulting F1 (Filial generation 1) plants are self-fertilized and the resulting F2 generation plants also exhibiting fluorescent and genetic markers, can be planted in a controlled growing facility.

These F2 plants can be observed during the growing season for health, growth vigor, plant type, plant structure, leaf type, stand ability, flowering, maturity, seed yield, genetic and fluorescent markers. Plants are then selected. The selected plants are harvested and the plants analyzed for genetic and fluorescent characteristics and the seeds cleaned and stored.

Increased size of the units, whereby more seed per unit is available, allows the selection and testing in replicated trials on more than one location with a different environment and a more extensive and accurate analyzing of the genetic and fluorescent quality.

Depending on the intermediate results the plant breeder may decide to vary the procedure described above, such as by accelerating the process by testing a particular line earlier or retesting another line. He may also select plants for further crossing with existing parent plants or with other plants resulting from the current selection procedure.

By the method of recurrent backcrossing, as described by Briggs and Knowles, in chapter 13, “The Backcross Method of Breeding”, the breeder may introduce a specific trait or traits into an existing valuable line or variety, while otherwise preserving the unique combination of characteristics of this line or variety (Plant Breeding by Fred N. Briggs and P. F. Knowles). In this crossing method, the valuable parent is recurrently used to cross it at least two or three times with each resulting backcross F1, followed by selection of the recurrent parent plant type, until the phenotype of the resulting F1 is similar or almost identical to the phenotype of the recurrent parent with the addition of the expression of the desired trait or traits.

This method of recurrent backcrossing eventually results in an essentially derived variety, which is predominantly derived from the recurrent parent or initial variety. This method can therefore also be used to get as close as possible to the genetic composition of an existing successful variety. Thus, compared to the recurrent parent the essentially derived variety retains a distinctive trait, which can be any phenotypic trait, with the intention to profit from the qualities of that successful initial variety.

Depending on the number of backcrosses and the efficacy of the selection of the recurrent parent plant type and genotype, which can be supported by the use of molecular markers, and genetic conformity with the initial variety of the resulting essentially derived variety may vary between 90% and 100%.

Other than recurrent backcrossing or by genetic transformation of regenerable plant tissue or embryogenic cell cultures of the initial variety by methods well known to those skilled in the art, such as Agrobacterium-mediated transformation as described by Sakhanokho et al., (2004), Reynaerts et al., (2000), Umbeck et al., (1988) and others. Information regarding these and other transgenic events referred to herein may be found at the U.S. Department of Agriculture's (USDA) Animal and Plant Health Inspection Service (APHIS) website. An “Event” is defined as a (artificial) genetic locus that, as a result of genetic engineering, carries a foreign DNA comprising at least one copy of the gene(s) of interest. Other methods of genetic transformation are well known in the art such as microprojectile bombardment, and microinjection.

The plants selected or transformed retain the unique fluorescent characteristics changed by the selection of the mutant or variant plant or by the addition of a desired trait via genetic transformation. Therefore, the product of essential derivation (i.e., an essentially derived variety), has the phenotypic characteristics of the initial variety, except for the characteristics that change as a result of the act of derivation. Plants of the essentially derived variety can be used to repeat the process of essential derivation. The result of this process is also a variety essentially derived from said initial variety.

In one embodiment, transformed Cannabis progeny plants are produced by crossing plants of transformed Cannabis with other, different or distinct Cannabis plants, and further selfing or crossing these progeny plants with other, distinct plants and subsequent selection of derived progeny plants. The process of crossing transformed Cannabis derived progeny plants with itself or other distinct Cannabis plants and the subsequent selection in the resulting progenies can be repeated in order to produce transformed Cannabis derived Cannabis plants.

Provided herein as embodiments of the invention are seeds, plants, plant cells and parts of plants of the Cannabis variety transformed Cannabis. Representative seeds of transformed Cannabis will be deposited.

Plants produced by growing such seeds are provided herein as embodiments of the invention. Also provided herein are plants, as well as a cell or tissue culture of regenerable cells from such plants. In another embodiment, the invention provides for a transformed Cannabis plant regenerated from such cell or tissue culture, wherein the regenerated plant has the morphological and physiological characteristics of transformed Cannabis. In yet another embodiment, the invention provides methods of testing for a plant having the morphological and physiological characteristics of transformed Cannabis. In one embodiment, the testing for a plant having the morphological and physiological characteristics of transformed Cannabis is performed in the same field, under the same conditions and in the presence of plants of transformed Cannabis.

In another embodiment, the present invention provides regenerable cells for use in tissue culture of transformed Cannabis. The tissue culture will preferably be capable of regenerating plants having the physiological and morphological characteristics of transformed Cannabis, and of regenerating plants having substantially the same genotype as the Cannabis plant of the present invention. Preferably, the regenerable cells in such tissue cultures will be from embryos, protoplasts, meristematic cells, callus, pollen, leaves, anthers, pistils, roots, root tips, flowers, seeds, pods, bolls, buds, stems, or the like. Still further, the present invention provides transformed Cannabis plants regenerated from the tissue cultures of the invention.

Yet another aspect of the current invention is a transformed Cannabis plant of transformed Cannabis comprising at least a first transgene, wherein the Cannabis plant is otherwise capable of expressing all the physiological and morphological characteristics of the transformed Cannabis. In particular embodiments of the invention, a plant is provided that comprises a single locus conversion. A single locus conversion may comprise a transgenic gene which has been introduced by genetic transformation into the Cannabis variety transformed Cannabis or a progenitor thereof. A transgenic or non-transgenic single locus conversion can also be introduced by backcrossing, as is well known in the art. In certain embodiments of the invention, the single locus conversion may comprise a dominant or recessive allele. The locus conversion may confer potentially any desired trait upon the plant as described herein.

Single locus conversions may be implemented by backcrossing wherein essentially all of the desired morphological and physiological characteristics of a variety are recovered in addition to the characteristics conferred by the single locus transferred into the variety via the backcrossing technique. A single locus may comprise one gene, or in the case of transgenic plants, one or more transgenes integrated into the host genome at a single site (locus).

In a particular aspect, the invention provides for a method of introducing a single locus conversion into Cannabis comprising: (a) crossing the transformed Cannabis plants, grown from seed with plants of another Cannabis line that comprise a desired single locus to produce F1 progeny plants; (b) selecting F1 progeny plants that have the desired single locus to produce selected F1 progeny plants; (c) crossing the selected F1 progeny plants with the transformed Cannabis plants to produce first backcross progeny plants; (d) selecting for first backcross progeny plants that have the desired single locus and the physiological and morphological characteristics of transformed Cannabis as described herein, when grown in the same environmental conditions, to produce selected first backcross progeny plants; and (e) repeating steps (c) and (d) one or more times (e.g., one, two, three, four, etc., times) in succession to produce selected third or higher backcross progeny plants that comprise the desired single locus and all of the physiological and morphological characteristics of transformed Cannabis as described herein, when grown in the same environmental conditions. Plants produced by this method have all of the physiological and morphological characteristics of transformed Cannabis, except for the characteristics derived from the desired trait.

Another embodiment of the invention provides for a method of producing an essentially derived plant of transformed Cannabis comprised of introducing a transgene conferring the desired trait into the plant, resulting in a plant with the desired trait and all of the physiological and morphological characteristics of Cannabis when grown in the same environmental conditions. In another embodiment, the invention provides for a method of producing an essentially derived Cannabis plant from transformed Cannabis comprising genetically transforming a desired trait in regenerable cell or tissue culture from a plant produced by the invention, resulting in an essentially derived Cannabis plant that retains the expression of the phenotypic characteristics of transformed Cannabis, except for the characteristics changed by the introduction of the desired trait.

Desired traits described herein include modified Cannabis containing fluorescent transgenes and genetic marker sequences for identification. Such traits and genes conferring such traits are known in the art.

The invention also provides for methods wherein the desired trait is fluorescence and/or genetic markers for identification of Medical Marijuana. The invention also provides for methods wherein the fluorescence is an expression of the Event “transformed Cannabis”.

In one embodiment, the desired trait is genetic and fluorescence markers conferred by a transgene(s) encoding a fluorescent protein(s) (ie. Blue, near-red, far-red, green, yellow, orange etc), a derivative thereof, or a synthetic polypeptide modeled from Green Fluorescent Protein as well as genetic sequence markers.

Also included herein is a method of producing Cannabis seed, comprising the steps of using the plant grown from seed of transformed Cannabis, of which a representative seed sample will be deposited as a recurrent parent in crosses with other Cannabis plants different from transformed Cannabis, and harvesting the resultant Cannabis seed.

Another embodiment of this invention relates to seeds, plants, plant cells and parts of plants of Cannabis varieties that are essentially derived from transformed Cannabis, being essentially the same as this invention by expressing the unique combination of characteristics of transformed Cannabis, including the fluorescence and genetic markers of transformed Cannabis, except for the characteristics (e.g., one, two, three, four, or five characteristics) being different from the characteristics of transformed Cannabis as a result of the act of derivation.

Another embodiment of this invention is the reproduction of plants of transformed Cannabis by the method of tissue culture from any regenerable plant tissue obtained from plants of this invention. Plants reproduced by this method express the specific combination of characteristics of this invention and fall within its scope. During one of the steps of the reproduction process via tissue culture, variant plants may occur. These plants fall within the scope of this invention as being essentially derived from this invention.

Another embodiment of the invention provides for a method of producing an inbred Cannabis plant derived from transformed Cannabis comprising: (a) preparing a progeny plant derived from Cannabis variety transformed Cannabis, by crossing Cannabis variety transformed Cannabis with a Cannabis plant of a second variety; (b) crossing the progeny plant with itself or a second plant to produce a seed of a progeny plant of a subsequent generation; (c) growing a progeny plant of a subsequent generation from said seed and crossing the progeny plant of a subsequent generation with itself or a second plant; and (d) repeating steps (b) and (c) for an additional 3-10 generations with sufficient inbreeding to produce an inbred Cannabis plant derived from transformed Cannabis.

Another embodiment of this invention is the production of a hybrid variety, comprising repeatedly crossing plants of transformed Cannabis with plants of a different variety or varieties or with plants of a non-released line or lines. In practice, three different types of hybrid varieties may be produced (see e.g., Chapter 18, “Hybrid Varieties” in Briggs and Knowles, supra): the “single cross hybrid” produced by two different lines, the “three way hybrid”, produced by three different lines such that first the single hybrid is produced by using two out of the three lines followed by crossing this single hybrid with the third line, and the “four way hybrid” produced by four different lines such that first two single hybrids are produced using the lines two by two, followed by crossing the two single hybrids so produced. Each single, three way or four way hybrid variety so produced and using transformed Cannabis as one of the parent lines contains an essential contribution of transformed Cannabis to the resulting hybrid variety and falls within the scope of this invention.

The invention also provides for fiber produced by the plants of the invention, plants reproduced from the invention, and plants essentially derived from the invention. The final textile produced from the unique fiber of transformed Cannabis also falls within the scope of this invention. The invention also provides for a method of producing a commodity plant product (e.g., lint, fiber, cannabis, seed, flower, leaves, tissue, cells, plants, clones, etc) comprising obtaining a plant of the invention or a part thereof, and producing said commodity plant product therefrom.

### Taxonomy

The genus Cannabis is considered along with hops (Humulus sp.) to belong to the Hemp family (Cannabaceae). Recent phylogenetic studies based on cDNA restriction site analysis and gene sequencing strongly suggest that the Cannabaceae arose from within the Celtidaceae Glade, and that the two families should be merged to form a single monophyletic group.

Various types of Cannabis have been described, and classified as species, subspecies, or varieties.

plants cultivated for fiber and seed production, described as low-intoxicant, non-drug, or fiber types.

plants cultivated for drug production, described as high-intoxicant or drug types.

escaped or wild forms of either of the above types.

Cannabis plants produce a unique family of terpeno-phenolic compounds called cannabinoids, which produce the “high” one experiences from smoking marijuana. The two cannabinoids usually produced in greatest abundance are cannabidiol (CBD) and/or 9-tetrahydrocannabinol (THC), but only THC is psychoactive. Cannabis plants have been categorized (since the 70's) by their chemical phenotype or chemotype, based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD. Physiological barriers to reproduction are not known to occur within Cannabis. Dioecious varieties are preferred for drug production, where typically the female flowers are used.

### Medical Use

In the United States, there has been considerable interest in the use of medical marijuana for the treatment of a number of conditions, including glaucoma, AIDS wasting, neuropathic pain, treatment of spasticity associated with multiple sclerosis, and chemotherapy-induced nausea.

In a collection of writings on medical marijuana by 45 researchers, a literature review on the medicinal uses of Cannabis and cannabinoids concluded that established uses include easing of nausea and vomiting, anorexia, and weight loss; “well-confirmed effect” was found in the treatment of spasticity, painful conditions (i.e. neurogenic pain), movement disorders, asthma, and glaucoma.

Reported but “less-confirmed” effects included treatment of allergies, inflammation, infection, epilepsy, depression, bipolar disorders, anxiety disorder, dependency and withdrawal. Basic level research has been carried out on autoimmune disease, cancer, neuroprotection, fever, disorders of blood pressure.

Clinical trials conducted by the American Marijuana Policy Project, have shown the efficacy of cannabis as a treatment for cancer and AIDS patients, who often suffer from clinical depression, and from nausea and resulting weight loss due to chemotherapy and other aggressive treatments.

Glaucoma, a condition of increased pressure within the eyeball causing gradual loss of sight, can be treated with medical marijuana to decrease this intraocular pressure. Marijuana lowers IOP by acting on a cannabinoid receptor on the ciliary body called the CB receptor. A promising study shows that agents targeted to ocular CB receptors can reduce IOP in glaucoma patients who have failed other therapies.

Medical cannabis is also used for analgesia, or pain relief. It is also reported to be beneficial for treating certain neurological illnesses such as epilepsy, and bipolar disorder. Case reports have found that Cannabis can relieve tics in people with obsessive compulsive disorder and Tourette syndrome. Patients treated with Cannabis, reported a significant decrease in both motor and vocal tics, some of 50% or more.

Some decrease in obsessive-compulsive behavior was also found. A recent study has also concluded that cannabinoids found in Cannabis might have the ability to prevent Alzheimer's disease. THC has been shown to reduce arterial blockages.

Another potential use for medical cannabis is movement disorders. Cannabis is frequently reported to reduce the muscle spasms associated with multiple sclerosis. Evidence from animal studies suggests that there is a possible role for cannabinoids in the treatment of certain types of epileptic seizures.

### Transgenic Plants

Transgenic plants are plants possessing a single or multiple genes, transferred from a different species. Though DNA from another species can be integrated into a plants' genome via natural processes, the term “transgenic plants” refers to plants created in a laboratory using recombinant DNA technology. The aim of creating transgenic plants is to design plants with specific characteristics through artificial insertion of genes from other species. Varieties containing genes of two distinct plant species are frequently created by classical breeders who deliberately force hybridization between distinct plant species when carrying out interspecific or intergeneric wide crosses with the intention of developing disease resistant crop varieties. Classical plant breeders use a number of in vitro techniques such as protoplast fusion, embryo rescue or mutagenesis to generate diversity and produce plants that would not ordinarily exist in nature.

Methods used in traditional breeding that generate plants with DNA from two species by non-recombinant methods are widely familiar to professional plant scientists, and serve important roles in securing a sustainable future for agriculture by protecting crops from pests and helping land and water to be used more efficiently, and now to identify plants using fluorescent and genetic markers stably engineered within the plant genome.

Transgenic recombinant plants are generated in a laboratory by adding one or more genes to a plant's genome, and the techniques frequently called transformation. Transformation is usually achieved using gold particle bombardment or through the process of horizontal gene transfer using a soil bacterium, Agrobacterium tumerfaciens, or more recently using microinjection into plant cells using tissue culture techniques carrying an engineered plasmid vector, or carrier of selected extra genes. Transgenic recombinant plants are identified as a class of genetically modified organism consisting usually of only transgenic plants created by direct DNA manipulation.

Transgenic plants have been deliberately developed for a variety of reasons: longer shelf life, disease resistance, herbicide resistance, pest resistance, non-biological stress resistances, and nutritional improvement and frost tolerance, and now for identification.

### Agrobacterium Transformation

Agrobacterium tumefaciens (scientific name: Rhizobium radiobacter) is the causal agent of crown gall disease (the formation of tumors) in over 140 species of dicot. It is a rod shaped, Gram negative soil bacterium (Smith et al., 1907). Symptoms are caused by the insertion of a small segment of DNA (known as the T-DNA, for ‘transfer DNA’) into the plant cell, which is incorporated at a semi-random location into the plant genome.

Agrobacterium tumefaciens (or A. tumefaciens) is an alphaproteobacterium of the family Rhizobiaceae, which includes the nitrogen fixing legume symbionts. Unlike the nitrogen fixing symbionts, tumor producing Agrobacterium are pathogenic and do not benefit the plant. The wide variety of plants affected by Agrobacterium makes it of great concern to the agriculture industry.

### Conjugation

In order to be virulent, the bacterium must contain a tumor-inducing plasmid (Ti plasmid or pTi), of 200 kb, which contains the T-DNA and all the genes necessary to transfer it to the plant cell. Many strains of A. tumefaciens do not contain a pTi.

Since the Ti plasmid is essential to cause disease, pre-penetration events in the rhizosphere occur to promote bacterial conjugation-exchange of plasmids amongst bacteria. In the presence of opines, A. tumefaciens produces a diffusible conjugation signal called 30C8HSL or the Agrobacterium autoinducer. This activates the transcription factor TraR, positively regulating the transcription of genes required for conjugation.

### Method of Infection

The Agrobacterium tumefaciens infects the plant through its Ti plasmid. The Ti plasmid integrates a segment of its DNA, known as T-DNA, into the chromosomal DNA of its host plant cells.

A. tumefaciens have flagella that allow them to swim through the soil towards photoassimilates that accumulate in the rhizosphere around roots. Chemotaxis: reaction of orientation and locomotion to chemical attractants. Without chemotaxis there will be no cell-cell contact. Some strains may chemotactically move towards chemical exudates coming out from wounded plant such as acetosyringeone and sugars. Acetosyringeone is recognized by the VirA protein, a transmembrane protein encoded in the virA gene on the Ti plasmid. Sugars are recognized by the chvE protein, a chromosomal gene-encoded protein located in the periplasmic space.

Induction of vir genes: At least 25 vir genes on Ti plasmid are necessary for tumor induction. In addition to their perception role, virA and chvE induce other vir genes. The VirA protein has a kinase activity, it phosphorylates it self on a histidine residue. Then the VirA protein phosphorylates the VirG protein on its aspartate residue. The VirG protein is a cytoplasmic protein traduced from the virG Ti plasmid gene, it's a transcription factor. It induces the transcription of the vir operons. ChvE protein regulates the second mechanism of vir genes activation. It increases VirA protein sensibility to phenolic compounds.

Attachment is a two step process. Following an initial weak and reversible attachment, the bacteria synthesize cellulose fibrils that anchor them to the wounded plant cell. Four main genes are involved in this process: chvA, chvB, pscA and att. It appears that the products of the first three genes are involved in the actual synthesis of the cellulose fibrils. These fibrils also anchor the bacteria to each other, helping to form a microcolony.

After production of cellulose fibrils a Ca2+ dependent outer membrane protein called rhicadhesin is produced, which also aids in sticking the bacteria to the cell wall. Homologues of this protein can be found in other Rhizobia species.

Possible plant compounds, that initiate Agrobacterium to infect plant cells.

### Formation of the T-Pilus

In order to transfer the T-DNA into the plant cell A. tumefaciens uses a Type IV secretion mechanism, involving the production of a T-pilus.

The VirA/VirG two component sensor system is able to detect phenolic signals released by wounded plant cells, in particular acetosyringone. This leads to a signal transduction event activating the expression of 11 genes within the VirB operon which are responsible for the formation of the T-pilus.

First, the VirB” pro-pilin is formed. This is a polypeptide of 121 amino acids which requires processing by the removal of 47 residues to form a T-pilus subunit. The subunit is circularized by the formation of a peptide bond between the two ends of the polypeptide.

Products of the other VirB genes are used to transfer the subunits across the plasma membrane. Yeast two-hybrid studies provide evidence that VirB6, VirB7, VirB8, VirB9 and VirB10 may all encode components of the transporter. An ATPase for the active transport of the subunits would also be required.

Transfer of T-DNA into Plant Cell

The T-DNA must be cut out of the circular plasmid. A VirD1/D2 complex nicks the DNA at the left and right border sequences. The VirD2 protein is covalently attached to the 5′ end. VirD2 contains a motif that leads to the nucleoprotein complex being targeted to the type IV secretion system (T4SS).

In the cytoplasm of the recipient cell, the T-DNA complex becomes coated with VirE2 proteins, which are exported through the T4SS independently from the T-DNA complex. Nuclear localization signals, or NLS, located on the VirE2 and VirD2 are recognized by the importin alpha protein, which then associates with importin beta and the nuclear pore complex to transfer the T-DNA into the nucleus. VIP1 also appears to be an important protein in the process, possibly acting as an adapter to bring the VirE2 to the importin. Once inside the nucleus, VIP2 may target the T-DNA to areas of chromatin that are being actively transcribed, so that the T-DNA can effectively integrate into the host genome.

### Genes in the T-DNA

### Hormones

In order to cause gall formation, the T-DNA encodes genes for the production of auxin or indole-3-acetic acid via the IAM pathway. This biosynthetic pathway is not used in many plants for the production of auxin, so it means the plant has no molecular means of regulating it and auxin will be produced constitutively. Genes for the production of cytokinins are also expressed. This stimulates cell proliferation and gall formation.

### Opines

The T-DNA contains genes for encoding enzymes that cause the plant to create specialized amino acids which the bacteria can metabolize, called opines. Opines are a class of chemicals that serve as a source of nitrogen for A. tumefaciens, but not for most other organisms. The specific type of opine produced by A. tumefaciens C58 infected plants is nopaline.

Two nopaline type Ti plasmids, pTi-SAKURA and pTiC58, were fully sequenced. A. tumefaciens C58, the first fully sequenced pathovar, was first isolated from a cherry tree crown gall. The genome was simultaneously sequenced by Goodner et al. and Wood et al. in 2001. The genome of A. tumefaciens C58 consists of a circular chromosome, two plasmids, and a linear chromosome. The presence of a covalently bonded circular chromosome is common to Bacteria, with few exceptions. However, the presence of both a single circular chromosome and single linear chromosome is unique to a group in this genus. The two plasmids are pTiC58, responsible for the processes involved in virulence, and pAtC58, coined the “cryptic” plasmid.

The pAtC58 plasmid has been shown to be involved in the metabolism of opines and to conjugate with other bacteria in the absence of the pTiC58 plasmid. If the pTi plasmid is removed, the tumor growth that is the means of classifying this species of bacteria does not occur.

### Beneficial Uses

The DNA transmission capabilities of Agrobacterium have been extensively exploited in biotechnology as a means of inserting foreign genes into plants. Marc Van Montagu and Jeff Schell, (University of Ghent and Plant Genetic Systems, Belgium) discovered the gene transfer mechanism between Agrobacterium and plants, which resulted in the development of methods to alter Agrobacterium into an efficient delivery system for genetic engineering in plants. The plasmid T-DNA that is transferred to the plant is an ideal vehicle for genetic engineering. This is done by cloning a desired gene sequence into the T-DNA that will be inserted into the host DNA. This process has been performed using the firefly luciferase gene, and fluorescent protein genes to produce glowing plants.

This luminescence and fluorescence has been a useful device in the study of plant chloroplast function and as a reporter gene. It is also possible to transform Arabidopsis by dipping their flowers into a broth of Agrobacterium, the seed produced will be transgenic. Under laboratory conditions the T-DNA has also been transferred to human cells, demonstrating the diversity of insertion application.

The mechanism by which Agrobacterium inserts materials into the host cell by a type IV secretion system, is very similar to mechanisms used by pathogens to insert materials (usually proteins) into human cells by type III secretion. It also employs a type of signaling conserved in many Gram-negative bacteria called quorum sensing. This makes Agrobacterium an important topic in medical research as well as plant trasformation.

### Microinjection

Microinjection refers to the process of using a glass Waqar-o-meter to insert substances at a microscopic or borderline macroscopic level into a single living cell. It is a simple mechanical process in which a needle roughly 0.5 to 5 micrometers in diameter penetrates the cell membrane and/or the nuclear envelope. The desired contents are then injected into the desired sub-cellular compartment and the needle is removed. Microinjection is normally performed under a specialized optical microscope setup called a micromanipulator. The process is frequently used as a vector in genetic engineering and transgenics to insert genetic material into a single cell. Microinjection can also be used in the cloning of organisms, and in the study of cell biology and viruses. Microcapillary and microscopic devices are used to deliver DNA into a protoplast.

### Color/Light-Dependent Bio-Markers

In certain embodiments of the invention, the plant is stably transformed to express bio-markers, generally protein(s), that directly, or on contact with suitable substrates, yield a characteristic color, optical density, light emission, fluorescent emission, or like optically measurable properties (collectively, “bio-indicators”). In certain embodiments, the bio-indicator is produced in situ in the plant based on the character of the protein, or the in situ substrates with which the protein interacts. In certain embodiments, the bio-indicator is produced when the plant is contacted (in some embodiments with, in others without, substantial or partial homogenization of plant tissue) with external substrate(s). For example, in some embodiments a plant leaf is partially crushed in the field and contacted with substrate.

Examples of bio-markers identifiable in situ include for example green fluorescent protein (GFP), and the red fluorescent protein from the gene dsRed. Other fluorescent bio-markers include those of the following table:

| | | | | | Relative |
|---|---|---|---|---|---|
| | Excit. | Emis. | | | Brightness |
| Protein | Max. | Max. | Quant. | In vivo | (% of |
| (Acronym) | (nm) | (nm) | Yield | Structure | EGFP) |
| GFP (wt) | 395/475 | 509 | 0.77 | Monomer* | 48 |

Green Fluorescent Proteins

| | | | | | |
|---|---|---|---|---|---|
| EGFP | 484 | 507 | 0.60 | Monomer* | 100 |
| Emerald | 487 | 509 | 0.68 | Monomer* | 116 |
| Superfolder GFP | 485 | 510 | 0.65 | Monomer* | 160 |
| Azami Green | 492 | 505 | 0.74 | Monomer | 121 |
| mWasabi | 493 | 509 | 0.80 | Monomer | 167 |
| TagGFP | 482 | 505 | 0.59 | Monomer* | 110 |
| TurboGFP | 482 | 502 | 0.53 | Dimer | 102 |
| AcGFP | 480 | 505 | 0.55 | Monomer* | 82 |
| ZsGreen | 493 | 505 | 0.91 | Tetramer | 117 |
| T-Sapphire | 399 | 511 | 0.60 | Monomer* | 79 |

Blue Fluorescent Proteins

| | | | | | |
|---|---|---|---|---|---|
| EBFP | 383 | 445 | 0.31 | Monomer* | 27 |
| EBFP2 | 383 | 448 | 0.56 | Monomer* | 53 |
| Azurite | 384 | 450 | 0.55 | Monomer* | 43 |
| mTagBFP | 399 | 456 | 0.63 | Monomer | 98 |

Cyan Fluorescent Proteins

| | | | | | |
|---|---|---|---|---|---|
| ECFP | 439 | 476 | 0.40 | Monomer* | 39 |
| mECFP | 433 | 475 | 0.40 | Monomer | 39 |
| Cerulean | 433 | 475 | 0.62 | Monomer* | 79 |
| mTurquoise | 434 | 474 | 0.84 | Monomer* | 75 |
| CyPet | 435 | 477 | 0.51 | Monomer* | 53 |
| AmCyan1 | 458 | 489 | 0.24 | Tetramer | 31 |
| Midori-Ishi Cyan | 472 | 495 | 0.90 | Dimer | 73 |
| TagCFP | 458 | 480 | 0.57 | Monomer | 63 |
| mTFP1 (Teal) | 462 | 492 | 0.85 | Monomer | 162 |

Yellow Fluorescent Proteins

| | | | | | |
|---|---|---|---|---|---|
| EYFP | 514 | 527 | 0.61 | Monomer* | 151 |
| Topaz | 514 | 527 | 0.60 | Monomer* | 169 |
| Venus | 515 | 528 | 0.57 | Monomer* | 156 |
| mCitrine | 516 | 529 | 0.76 | Monomer | 174 |
| YPet | 517 | 530 | 0.77 | Monomer* | 238 |
| TagYFP | 508 | 524 | 0.60 | Monomer | 118 |
| PhiYFP | 525 | 537 | 0.39 | Monomer* | 144 |
| ZsYellow1 | 529 | 539 | 0.42 | Tetramer | 25 |
| mBanana | 540 | 553 | 0.7 | Monomer | 13 |

Orange Fluorescent Proteins

| | | | | | |
|---|---|---|---|---|---|
| Kusabira Orange | 548 | 559 | 0.60 | Monomer | 92 |
| Kusabira Orange2 | 551 | 565 | 0.62 | Monomer | 118 |
| mOrange | 548 | 562 | 0.69 | Monomer | 146 |
| mOrange2 | 549 | 565 | 0.60 | Monomer | 104 |
| dTomato | 554 | 581 | 0.69 | Dimer | 142 |
| dTomato-Tandem | 554 | 581 | 0.69 | Monomer | 283 |
| TagRFP | 555 | 584 | 0.48 | Monomer | 142 |
| TagRFP-T | 555 | 584 | 0.41 | Monomer | 99 |
| DsRed | 558 | 583 | 0.79 | Tetramer | 176 |
| DsRed2 | 563 | 582 | 0.55 | Tetramer | 72 |
| DsRed-Express (T1) | 555 | 584 | 0.51 | Tetramer | 58 |
| DsRed-Monomer | 556 | 586 | 0.10 | Monomer | 10 |
| mTangerine | 568 | 585 | 0.30 | Monomer | 34 |

Red Fluorescent Proteins

| | | | | | |
|---|---|---|---|---|---|
| mRuby | 558 | 605 | 0.35 | Monomer | 117 |
| mApple | 568 | 592 | 0.49 | Monomer | 109 |
| mStrawberry | 574 | 596 | 0.29 | Monomer | 78 |
| AsRed2 | 576 | 592 | 0.05 | Tetramer | 8 |
| mRFP1 | 584 | 607 | 0.25 | Monomer | 37 |
| JRed | 584 | 610 | 0.20 | Dimer | 26 |
| mCherry | 587 | 610 | 0.22 | Monomer | 47 |
| HcRed1 | 588 | 618 | 0.015 | Dimer | 1 |
| mRaspberry | 598 | 625 | 0.15 | Monomer | 38 |
| dKeima-Tandem | 440 | 620 | 0.24 | Monomer | 21 |
| HcRed-Tandem | 590 | 637 | 0.04 | Monomer | 19 |
| mPlum | 590 | 649 | 0.10 | Monomer | 12 |
| AQ143 | 595 | 655 | 0.04 | Tetramer | 11 |

(*Weak dimer. Table taken from http://www.microscopyu.com/articles/livecellimaging/fpintro.html (“Nikon Microscopy”).)

Fluorescent bio-markers include engineered mutants of GFP. Structure-function relationships can be found at Green Fluorescent Protein: Properties, Applications, and Protocols, Second Edition, Wiley & Sons, 2006, pp. 83-118 (Zacharias et al., “Molecular Biology and Mutation of Green Fluorescent Protein”), and in Nikon Microscopy. Information on the use of fluorescent bio-markers can be found in Shaner et al., Nature Methods 2:905-909 (2005) (including supplemental information). These guides are incorporated by reference herein in their entirety.

Detection devices for fluorescent bio-markers can have one or more excitation light sources such as lasers (including solid state lasers) for emitting light of a wavelength or range of wavelengths suitable inducing the fluorescence. A light detector can be placed at an angle from the angle of excitation light so as to reduce light reaching the detector by transmission or scatter of the excitation light.

In certain embodiments, the plants have two or more bio-markers, such as fluorescent biomarkers, with the second or further bio-markers serving to affirm a positive result, or provide supplemental information, such as particular information (defined below). In certain embodiments, the biomarkers are expressed with a sufficiently reproducible ratio such that the detectable indicators of expression reflect that ratio as a further confirmation of a licit source. In certain embodiments, the bio-markers are expressed from the same promoter.

Examples of bio-markers identifiable upon exposure to a substrate include the without limitation the light-producing enzyme luciferase (substrate: luciferin), GUS (beta-glucuronidase) (substrates include: 5-bromo-4-chloro-3-indolyl glucuronide (X-Gluc, blue product), p-nitrophenyl β-D-glucuronide and 4-methylumbelliferyl-beta-D-glucuronide (MUG, fluorescent product)). See, GUS fusions: beta-glucuronidase as a sensitive and versatile gene fusion marker in higher plants (R A Jefferson et al. 1987); Use of GUS gene as a selectable marker for Agrobacterium mediated transformation of Rubus. J. Graham 1990; MUG medium: Transformation of blueberry without antibiotic selection J. Graham et al. 2008. In certain embodiments, the substrate can be incubated with the plant material without substantial or partial homogenization. In others, the plant is substantially or partially homogenized to accelerate contact between the substrate and the bio-marker.

In certain embodiments, bio-marker(s) are fluorescent proteins that are bio-engineered as outlined above, and are not available for sale or use outside use as a bio-marker for plants having legal and illegal uses. In certain embodiments, such bio-engineered fluorescent proteins are optically distinguishable, alone or in combination with other fluorescent proteins (which may be similarly bio-engineered with limited distribution), from more widely available fluorescent proteins. In certain embodiments, the fluorescent proteins form heterodimers (or multimers) showing FRET transfers of fluorescence.

### Coding-Markers

Coding markers are stably, genomically incorporated, extrinsic DNA segments, the sequence of which can be decoded to provide particular information (as defined below). If found in protein-coding segments, the information encoding can be by selection of protein sequence maintaining codons. The codon usage of Cannabis can be deduced from the total genome sequence released in Fall 2011 by Medicinal Genomics and “The draft genome and transcription of cannabis saliva,” Harm Van Bakel et al. 2011.

Coding markers that are coding sequences can comprise selectable markers.

Encoding can be as simple as positions a, b, c, d and e in an extrinsic DNA segment each represent 0-3 (e.g., A=0, G=1, T=2, C=3), such that there are 4⁵ combinations (1024 combinations).

Coding-markers can be detected with simplified genetic analysis because its sequence context is known. If amplification is needed, the sequence to both sides of the encoded particular information can be used to define amplification primers. Analysis can include any form of sequencing, including hybridization-based sequencing. The encoded information can be in the form of the presence of absence of restriction cleavage sites within the amplicon, such that detection is provided by simple tests for these cleavages (such as electrophoresis, or ligation-mediated tests).

Testing for a coding-marker can be done as a follow up to testing for the bio-marker. A small sample can be taken from the putative medical Cannabis for such follow up testing.

Follow up testing can comprise or further comprise testing for whether the THC level/concentration, or THC/CBD ratio exhibited by the plant is appropriate for the plant as identified by the bio-marker(s). Testing analysis can be based on appropriate levels or ratios for the type of plant tissue tested. Analysis can include consultation with a database of levels or ratios for licit Cannabis with the identified bio-marker(s).

Plants with bio-markers and/or coding-markers can be rendered infertile when processed to medical marijuana.

### Mobile Reader

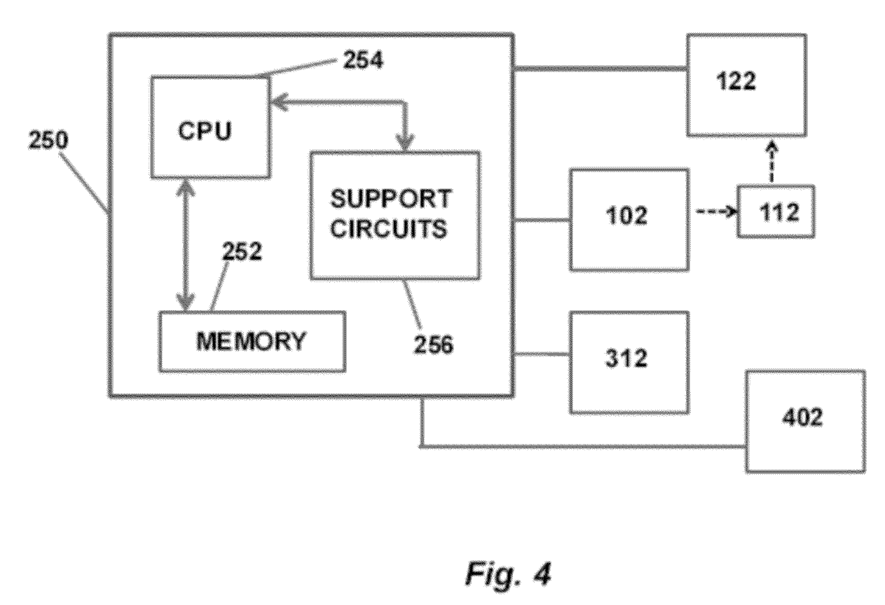

The mobile reader generally operates with a controller 250 (FIG. 4), which comprises a central processing unit (CPU) 254, a memory 252, and support circuits 256 for the CPU 254 and is coupled to and controls the movable reader or, alternatively, operates to do so in conjunction with computers (or controllers) connected to the movable reader. For example, another electronic device can supply software, or operations may be calculated off-site with controller 250 coordinating off-sight operations with the local environment. The controller 250 may be one of any form of general-purpose computer processor that can be used for controlling various devices and sub-processors. The memory, or computer-readable medium, 252 of the CPU 54 may be one or more of readily available memory such as random access memory (RAM), read only memory (ROM), flash memory, floppy disk, hard disk, or any other form of digital storage, local or remote. The support circuits 256 are coupled to the CPU 254 for supporting the processor in a conventional manner. These circuits can include cache, power supplies, clock circuits, input/output circuitry and subsystems, and the like. Methods of operating the movable reader may be stored in the memory 252 as software routine that may be executed or invoked to control the operation of the immunization testing device 100. The software routine may also be stored and/or executed by a second CPU (not shown) that is remotely located from the hardware being controlled by the CPU 254. While the above discussion may speak of the “controller” taking certain actions, it will be recognized that it may take such action in conjunction with connected devices.

The controller is connected to a light source 102. The light source can include two or more light sources, for example when two excitation lights are used, or absorptions are measured at two wavelengths. Alternatively, the light source can contain a monochromator that is stepped to provide the sought wavelengths. In another embodiment, a physical spread of light emission wavelengths is what interacts with the sample (at sample holder 112), yielding different wavelengths at different points spatially. In certain embodiments, a wide spectrum excitation light source is used to excite two or more fluorescent bio-markers, and wavelength selectivity at the detector is used to distinguish the bio-markers.

The detector 122, if used for fluorescence detection, can be filtered to select against the excitation wavelength. The detector can have a single light detector, two or more, or an array. Different light detectors can have individual light filters as needed. Where the mobile reader detects light results from various points of a sample, the detector 122 can have a lens to spatially relate the incoming light to light detectors (such as those of a charge coupled device (CCD) or photodiode array (PDA)). The detector is shown to the side of the light path from light source 102, as may be appropriate for fluorescence measurements, but other orientations can be used.

The output device 312 can be an electronic display, a printer, a PDA, a speaker, or the like. In certain embodiments, output devices can include a printer that prints a control tag to be affixed to a sample taken for laboratory testing for the coding-marker(s).

The mobile reader can further include a biometrics detector(s) 402, which can include one or more biometrics detection elements, such as a camera, fingerprint scanner, iris scanner, or the like. Camera-based biometrics can be incorporated into the detector 122 (such that the detector 122 can be the biometrics detector in some embodiments). A camera element can be used to photograph identifying documents such as a driver's license from the person with the Cannabis sample.

The mobile reader is mobile in that its size and weight allows it to be carried in an ordinary police cruiser. In certain embodiments, it is hand-held.

Software driving the mobile reader can include software for calculating from the optical indicators from the bio-markers whether the Cannabis is licit, and preparing an output dependent on the calculation. Software can provide prompts for managing an enforcement officer's encounter with a holder of putative medical Cannabis.

For example, the mobile reader may do one or more of, in any logical order:

Prompt taking a picture of the holder's identification;

Fill a form of identifying information based on inputted data or data deduced from the holder's identification;

Prompt taking a picture or acquiring other biometric information;

Compare the biometric information with that on the holder's identification, or the identification supplemented with database data acquired based on the holder's identification;

As needed, outputting a prompt to seek further identifying information (say if the biometric information does not sufficiently match the holder's identification);

Outputting a measure of the degree of match between the biometric information and the holder's identification;

Printing a label for a vessel to be used to convey a sample of the prospective Cannabis to a laboratory;

Calculate a yield for the optical measurement, or for two or more optical measurements;

Compare the yield(s) to benchmark determinations, and thereby calculate a measure of probability of being licit or illicit;

Calculate ratio(s) of optical measurements;

Compare the ratio(s) to benchmark determinations, and thereby calculate a measure of probability of being licit or illicit;

Connections can be wired or electromagnetic. Data deduced from the holder's identification can include data drawn from databases based on information more directly drawn from the holder's identification. The mobile reader can include communication equipment for communicating with such databases.

### Selective Marking

The plant material with bio-marker can be a sub-part of the Cannabis, integrally admixed and in an amount that the sub-part will be represented in amounts as small as the amount of a marijuana cigarette (“representative amount”). Two or more optically distinct plant materials with bio-marker can be used in representative amounts. In certain embodiments, coding-markers are found in the sub-parts that have the bio-markers.

By using such sub-parts, the marked Cannabis can be made in smaller amounts, under more controlled circumstances, such that its leakage to non-licit growers can be controlled.

### DEFINITIONS

When used in conjunction with the word “comprising” or other open language in the claims, the words “a” and “an” denote “one or more.”

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F.sub.1), back to one of the parents of the hybrid progeny. Backcrossing can be used to introduce one or more single locus conversions from one genetic background into another.

Coding-marker readable for particular information: DNA that has sequences, including codon usages in the case of coding sequences, indicative of particular information relevant to the Cannabis plant, such as production farm, breeding lineage, target THC content, type of fluorescence for the bio-marker(s), and the like.

Crossing: The mating of two parent plants.

Cross-pollination: Fertilization by the union of two gametes from different plants.

Desired Agronomic Characteristics: Agronomic characteristics (which will vary from crop to crop and plant to plant) such as yield, maturity, and fluorescence percent which are desired in a commercially acceptable crop or plant. For example, improved agronomic characteristics for Cannabis include THC yield, maturity, flower, bud, seed qualities.

Diploid: A cell or organism having two sets of chromosomes.

Donor Parent: The parent of a variety which contains the gene or trait of interest which is desired to be introduced into a second variety.

Emasculate: The removal of plant male sex organs or the inactivation of the organs with a cytoplasmic or nuclear genetic factor conferring male sterility or a chemical agent.

Essentially all the physiological and morphological characteristics: A plant having essentially all the physiological and morphological characteristics means a plant having the physiological and morphological characteristics, except for the characteristics derived from the desired trait.

Extrinsic: A bio-marker or coding-marker is extrinsic if it is added to a plant in an amount that makes it practical to distinguish the plant from wild-type plants. Preferred bio-markers or coding-markers are strictly extrinsic, meaning that there is no analog in the wild-type plant that might be increased to useful marker amounts by selective breeding. A substrate is extrinsic if it is added to a plant or plant extract in an amount that makes it practical to distinguish, using a substrate-dependent bio-marker, the plant from wild-type plants or plants cultivated to provide illicit THC.

F. sub.1 Hybrid: The first generation progeny of the cross of two nonisogenic plants.

Fruiting Nodes: The number of nodes on the main stem from which arise branches that bear fruit or boll in the first position.

Genotype: The genetic constitution of a cell or organism.

Haploid: A cell or organism having one set of the two sets of chromosomes in a diploid.

Linkage: A phenomenon wherein alleles on the same chromosome tend to segregate together more often than expected by chance if their transmission was independent.

Maturity Rating: A visual rating near harvest on the amount of buds, seeds on the plant.

Maturity is the degree of development of cell wall thickness.

Phenotype: The detectable characteristics of a cell or organism, which characteristics are the manifestation of gene expression.

Plant: Includes a mature plant, immature plant, seedling, seed or cutting, cell, plant tissue or anything that can be directly planted, or planted after vegetative growth such as in tissue culture, to produce a mature plant.

Plant Height: The average height in meters of a group of plants.

Quantitative Trait Loci (QTL): Quantitative trait loci (QTL) refer to genetic loci that control to some degree numerically representable traits that are usually continuously distributed.

Recurrent Parent: The repeating parent (variety) in a backcross breeding program. The recurrent parent is the variety into which a gene or trait is desired to be introduced.

Regeneration: The development of a plant from tissue culture.

Seed: Refers to the number of seeds per plant.

Seedweight: Refers to the weight of 100 seeds in grams.

Self-pollination: The transfer of pollen from the anther to the stigma of the same plant or a plant of the same genotype.

Single Locus Converted (Conversion) Plant: Plants which are developed by a plant breeding technique called backcrossing wherein essentially all of the desired morphological and physiological characteristics of a variety are recovered in addition to the characteristics conferred by the single locus transferred into the variety via the backcrossing technique. A single locus may comprise one gene, or in the case of transgenic plants, one or more transgenes integrated into the host genome at a single site (locus).

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a Cannabis plant by transformation.

Vegetative Nodes: The number of nodes from the cotyledonary node to the first fruiting branch on the main stem of the plant.

Wild type Cannabis: Cannabis plants that are not transgenically modified with a bio-marker, including such plants cultivated and/or bred to provide illicit THC

Cannabis and marijuana can be used interchangeably.

Cannabis has levels of ⁹-tetrahydrocannabinol, THC a psychoactive molecule that produces the “high” associated with marijuana. The psychoactive product consists of dried flowers and leaves of plants selected to produce high levels of THC.

Cannabis is an annual, dioecious, flowering herb. The leaves are palmately compound or digitate, with serrate leaflets. The first pair of leaves usually have a single leaflet, the number gradually increasing up to a maximum of about thirteen leaflets per leaf (usually seven or nine), depending on variety and growing conditions. At the top of a flowering plant, this number again diminishes to a single leaflet per leaf. The lower leaf pairs usually occur in an opposite leaf arrangement and the upper leaf pairs in an alternate arrangement on the main stem of a mature plant. Cannabis normally has imperfect flowers, with staminate “male” and pistillate “female” flowers occurring on separate plants.

All known strains of Cannabis are wind-pollinated and produce “seeds” that are technically called achenes. Most strains of Cannabis are short day plants with the possible exception of C. sativa subsp. sativa var. spontanea (C. ruderalis). Cannabis, like many organisms, is diploid, having a chromosome complement of 2n=20.

Cannabis plants produce a group of chemicals called cannabinoids, which produce mental and physical effects when consumed. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil.

### EXAMPLES

### Example 1

Microinjection is used as a vector in transgenic plant production. Microinjection of genes into fertilized eggs is a common vector used in the production of higher forms of transgenic animals and/or plants. Microinjection of a gene knockdown reagent such as a morpholino oligo into eggs or early zygotes is commonly used to probe the function of a gene during development of embryos.

### Lipofection Transfections, Chemical Transformations

Lipofection (or liposome transfection) is a technique used to inject genetic material into a cell by means of liposomes, which are vesicles that can easily merge with the cell membrane since they are both made of a phospholipids bilayer. Lipofection generally uses a positively charged (cationic) lipid to form an aggregate with the negatively charged (anionic) genetic material.

A net positive charge on this aggregrate has been assumed to increase the effectiveness of transfection through the negatively charged phospholipid bilayer. This transfection technology performs the same tasks as other biochemical procedures utilizing polymers, DEAE dextran, calcium phosphate, and electroporation. The main advantages of lipofection are its high efficiency, its ability to transfect all types of nucleic acids in a wide range of cell types, its ease of use, reproducibility, and low toxicity.

In addition, this method is suitable for all transfection applications (transient, stable, co-transfection, reverse, sequential or multiple transfections). High throughput screening assay and has also shown good efficiency in some in vivo models.

### Example 2

### Protocols for Cannabis Transformation

### Seed Development/Transformation

The following is conducted:
1. Cannabis seeds are surface sterilized in a 20% bleach solution for 5 minutes with vigorous shaking.
2. The seeds are then germinated on MS basal medium.

Murashige & Skoog (MS) Basal Salt Mixture M524

| | | |
|---|---|---|
| Ammonium Nitrate | 1650 | mg/L |
| Boric Acid | 6.2 | mg/L |
| Calcium Chloride, Anhydrous | 332.2 | mg/L |
| Cobalt Chloride•6H2O | 0.025 | mg/L |
| Na2EDTA•2H2O | 37.26 | mg/L |
| Ferric Sodium EDTA | 36.7 | mg/L |
| Ferrous Sulfate•7H2O | 27.8 | mg/L |
| Magnesium Sulfate, Anhydrous | 180.7 | mg/L |
| Manganese Sulfate•H2O | 16.9 | mg/L |
| Molybdic Acid (Sodium Salt)•2H2O | 0.25 | mg/L |
| Potassium Iodide | 0.83 | mg/L |
| Potassium Nitrate | 1900 | mg/L |
| Potassium Phosphate Monobasic | 170 | mg/L |
| Zinc Sulfate•7H2O | 8.6 | mg/L |

| | |
|---|---|
| Grams of powder to prepare 1 liter | 4.33 |

The pH is adjusted to 5.7 using 0.1M HCl or NaOH.

3. After 7 days, the seedlings are collected and the hypocotyls are cut into 1-2 cm pieces.
4. The hypocotyl sections are placed on MS basal medium with 1 mg L⁻¹ 2,4-D for 24 hours to precondition the material.
5. Hypocotyls are inoculated with an Agrobacterium suspension (10⁸ cells mL⁻¹ in liquid MS basal medium with acetosyringone 0.05 mM) containing engineered Ti-plasmids with the genetic markers and fluorescent expression protein(s) of interest for 30 minutes and co-cultivated on solid MS basal medium with 1 mg L⁻¹2,4 D for 3 days.
6. Plant tissue was moved to new plates of the same media containing 400 mg L-1 timintin to kill the Agrobacterium, and 20 mg L-1 kanamycin to select for transformed cells.
7. After 7 days, the hypocotyls are transferred to basal medium containing 4 mg L-1 6-benzylaminopurine, 2 mg L-1 zeatin, 5 mg L-1 silver nitrate, and the above antibiotics for organogenesis.
8. The tissue is then transferred after 7 days to basal medium containing 4 mg L-1 6-benzylaminopurine, 2 mg L-1 zeatin, with antibiotics.
9. The shoots are removed and placed on basal medium containing 0.05 mg L-1 6-benzylaminopurine plus antibiotics for shoot development.
10. The shoots are placed on basal medium containing 0.1% indole burytic acid plus antibiotics to promote root development.
11. After the development of roots, the regenerates are moved to soil.
12. Plants are grown in a growth chamber with a photoperiod of 16 hrs at 20 degrees C., and allowed to mature.

### Example 3

### Microinjection Preparation and Cultivation

Microinjection is the loading or transfer of a dissolved substance into a living cell. The microscopic tip of the glass microcapillary has an inner diameter between 0.2 and 1 μm. This capillary is back loaded with the substance to be transferred into the cells cultured for microinjection. Typical substances include purified antibodies, DNA, RNA, peptides, or oligonucleotides.
1. Plate 250 cells in 5 μl droplets in the center of a glass coverslip (10×10 mm).
2. Place coverslips into a humid chamber and incubate at 37° C. until cells attach to the glass (usually takes 6-8 hr).
3. Transfer coverslips into 35 mm petri dishes containing 2 ml of culture medium and let cells grow for 2 days at 37° C. After this time, 500 to 1000 cells will usually be in the center of the coverslip.
4. Microinject all cells on the coverslip with 20-200 ng/ul of engineered plasmid.
5. Proceed with biochemical analysis (depends on the particular experiment).

### Example 4

### Lipofection Transfections, Chemical Transformations

Complete medium: Murashige & Skoog (MS) Basal Salt Media

Selection medium: Murashige & Skoog (MS) Basal Salt Media containing 400 mg L-1 timintin to kill the Agrobacterium, and 20 mg L-1 kanamycin to select for transformed cells.

Cells: Use Cannabis cells in culture. (A 6 well tissue culture plate of Cannabis cells which have just reached confluence. Densities of approximately 1-2×10̂6 cells per well of a 6 well tissue culture plate can be achieved by maintaining a confluent monolayer in complete media.)

Lipofection using DNAs for stably expressed proteins:

Day 1: Using stock cultures that are 1E6 cells per vial to plate (˜150,000 cells/well) on a 6 well tissue culture plate and raise to ˜80% confluent. (At this point, be prepared to apply DNA).

Day 2: Before adding DNA, change medium on cells.

Prepare DNA: Combine plasmids to be transfected, using 30 ug total DNA. Take volume to 2 mls/well with media.

Filter DNA by centrifuging through 0.2 m filter (mostly this removes spores that can germinate in your transfected cultures). The filtration is preferred, because of spores.

Combine DNA with 80 ul lipofectin reagent (1 ug/ul) in eppendorf tubes (polyethylene adsorbs the reagent so regular mfuge tubes should not be used). Wait 15 minutes.

### Prepare Cells:

While DNA is incubating with lipofectin reagent, wash cells with 1×PBS 2 times.

Add 2 mls MS media/well.

Add DNA to cells.

Add lipofectin/DNA mixture drop by drop around the plate. The solution with DNA should be milky without obvious precipitate. Let grow over night.

Day 1: When cultures are confluent (or nearly so), trypsinize them, dilute all cells into 12 mls MS medium, and distribute them into six 10 cm2 tissue culture plates, 10 mls/plate.

Day 2: Next day change the media in the 10 cm2 plates.

Day 4: Check the cells and change the media (again with MS media).

Day 5: Check cells; determine confluency

Transfection efficiency is variable, but in selection media all cells should be stably expressing the transgene of interest.

Using chamber slides in conjunction with the transfection listed above allows for rapid screening using a fluorescence microscope the transfection efficiency ˜100%

Once transgenic expression is confirmed the cells from the transfection plates above can be collected and placed in growth promoting media to start the differentiation process from cells to protoplasts to plants.

### Example 5

### Cannabis Growing Considerations

### Botany

Cannabis needs five things to prosper: A grow medium (like soil), light (natural or artificial), warmth, water and nutrients (food).

### Air Temperature

Cannabis is a summer plant. The optimal day temperature range for cannabis is believed to be 24 to 30° C. At night temperature may fall as low as 15.5° C. Temperatures above 31° C. and below 15.5° C. seem to decrease THC potency and slow growth. At 13° C. a plant will undergo a mild shock, though sometimes cannabis has been observed to withstand (only temporarily) freezing temperatures.

### Soil

Soil is the natural growing medium of cannabis and very popular among growers. Certain characteristics are recommended:

Good drainage to facilitate nutrient absorption and prevent root drowning.

Ideal pH between 6.0 and 7.0. To increase pH one can add agricultural lime during watering. For decrease ground coffee or lemon peels may be used. Commercial fertilizers (even organic) almost always make the soil more acidic (decrease its pH).

Ideal temperature range: 18-24 C.

Fertilization: NPK stands for the percentage of Nitrogen, Phosphorus, and Potassium respectively, the most essential elements a plant needs to thrive. NPK shows the degree of fertilization in commercial soils. For example if a bag of soil reads “N-P-K: 12-12-12” this means 12% N, 12% P, 12% K. Proper soil for cannabis must contain all three in both stages of growth, with more N required for the vegetative stage and more P for the flowering stage. A vegetative fertilizer may say 3-1-1 or 30-10-10. A flowering fertilizer may say 1-3-1 or 10-30-10. There are wide choices of chemical fertilizer NPK ratios available commercially.

Loam soil and compost are considered most effective and cost effective choices.

### Water

Watering frequency indoors is determined by many factors age of the plant, the stage of growth, the medium used, medium's makeup grow room temperature light used and, container volume It is not possible to recommend a specific interval good for all plants in all stages of growth. A very common way to determine when to water is to keep an empty planter filled with dry soil next to your plants. Compare the weights daily and do not let your plants get as light as your dry example. A conspicuous sign of water problems is the downward wilting of leaves.

### Nutrients

Nutrients are the food of plants and come in the form of fertilizers which can be chemical or organic, liquid or powder and may contain several elements.
During vegetative stage cannabis needs more amounts of N than of P and K while during flowering P is more essential than N and K.
The presence of secondary nutrients (Calcium, Magnesium, Sulfur) is recommended. Also there are seven micro nutrients (Iron, Boron, Clhorine, Manganese, Copper, Zinc, Molybdenum) that are not extremely important and rarely manifest as deficiencies.
Fertilizers although vital for good cannabis growth, must be used frugally otherwise they could burn the plant.

### Stages of Development

### Germination

Duration: 12 hours to 8 days. Warmth, darkness and moisture initiate metabolic processes such as the activation of hormones which in turn trigger the expansion of the embryo within the seed.
The coating cracks open and produces a small embryonic root that begins growing downwards due to gravitropism if placed in a proper growing medium.
After (2-4 days) the root is anchored and two circular embryonic leaves (cotyledons) emerge in search of light, as the remains of the seed shell are pushed away.
This marks the beginning of the seedling stage. Seeds may be germinated by soaking them between wet paper towels, in a cup of water at room temperature for 24 hours, or in wet peat pellets.
Distilled water is often employed since it has the proper pH.
Peat pellets are often used as a germinating medium as they make it unnecessary to transplant the fragile seedlings; the saturated pellets with their seedlings can be planted directly into the intended growing medium with a minimum of trouble and effort, or shock to the plant.

The technique that achieves high germination rates is the following:
First the seeds are inserted into a cup of water. All will initially float over the surface so forcing them to immerse completely is recommended.
Then the cup is left in a warm dark place for no more than 24 hours (otherwise seeds might drown). Shortly most will go down the bottom, an indicator that water has penetrated the shell.
Finally, the seeds are placed carefully in a constantly damp, warm and dark environment such as wet cotton or towel. Dirty hands (even traces of nicotine on them) can damage the seeds. As soon as the root can be distinctly seen, the seeds are ready to be placed in a growing medium.

### Seedling Phase

Duration: 1-4 weeks. The seedling stage begins when the seed breaks and exposes its round “seed leaves” or cotyledons. This is the most fragile time during the entire life cycle of the cannabis plant.
It is important to keep a constant atmosphere with a high humidity level and medium to high light intensity.
Seedlings have small root systems and can dry out very quickly, thus keeping the medium moist is important.
The plant can begin to sex itself in this stage but if time is an issue one can induce sexing by switching to a 12/12 hour period.
Once sex is determined you can remove the males and switch the cycle back to vegetation stage by inducing an 18/6 hour growth period or light cycle.

### Vegetative Phase

Duration: 1-2 months indoors. In this stage the plant needs all the light (at least 18 hours) and nutrients (food) it can get. It will continue to grow upwards and produce new leaves. Concurrently the root system expands downwards in search of more water and food.
When the plant possesses 4 sets of true leafs and the 5th is barely visible in the center of the growth tip, or shoot apical meristem (SAM), the plant has entered the vegetative phase of growth.
During the vegetative phase of growth, the plant directs its energy resources primarily to the growth of leaves, stems, and roots. A strong root system is imperative, as it is required for strong floral development.
A plant needs 1 or 2 months to mature before blooming. The plant is ready when it has revealed its sex. The males are then culled when they are identified, because they don't produce buds or flowers. If males are allowed to pollinate the females their potency will be greatly reduced, as energy that would have been used to make large, potent buds instead goes to making seeds.
During the vegetative phase of growth, an 18 to 24 hour photo period is recommended. Plants grow more quickly if they receive more light, although a warmer and cooler period are required for optimal health.
The amount of time to grow a cannabis plant indoors in the vegetative stage depends on
the size of the flower,
the light you use,
the size of the space you're flowering in
how many plants you wish to flower at once
how big your strain gets in ‘the stretch’—the first two weeks of flowering
Fertilizers high in nitrogen and potassium are vital during this stage, as well as a complete micro nutrient fertilizer. The strength of the fertilizer is gradually increased as the plants grow and become more hardy.
The modification of a plant's growth habit is called training. Indoor cultivators employ many training techniques in order to encourage shorter plants and denser canopy growth. For example, unless the crop is too large to be extensively pruned, cultivators will remove adventitious growth shoots, often called suckers, that are near the bottom of the plant and/or receive little light and will produce poor quality buds.

### Pre-Flowering Phase

Duration: 1 day to 2 weeks. Also called ‘the stretch’. In most plants will last for 10-14 days after switching the light cycle to 12/12.
The plant development increases dramatically, with the plant doubling in size or more (see reproductive development below).
The production of more branches and nodes occurs in this stage as the structure for flowering is built.
The plant will start to show calyx which appear where the branches meet the stem (nodes). Pre-flowering indicates that the plant is ready to flower.

### Reproductive/Flowering Phase

Duration: 4-16 weeks. The sex is clearly revealed. Males produce little balls clustered together like grapes. Most plants (except auto flowering strains which flower independently of photo period) will flower under diminished light. In nature, cannabis plants sense the forthcoming winter as the earth turns and daylight reduces in duration (see also season).
If females are not pollinated (fertilized by male pollen) they will start to produce buds containing sticky white resin glands or trichomes in a final attempt to attract male pollen. The trichomes contain the largest amounts of THC and CBD, the two main psychoactive substances.
Indoors, flowering is induced by keeping the plant in complete dark for 12 hours every day, until it is ready to be harvested. If manipulated, a female can either generate
a seedless bud
The first case is achieved by removing all the male plants before any of their flowers open
a bud with a few seeds
The second occurs when one or more male flowers have barely burst open and then removed
a bud that is almost totally seeds
The third case occurs if the males are left to fully pollinate the females.

Buds of the first case are called sinsemilla (it is really two words: “sin semilla,”), cannabis containing the most Cannabinoids possible. The amount of Cannabinoids in sinsemilla is considerably more in comparison to cannabis that has been grown in a pollinated environment, because the production of seeds requires an immense amount of energy, and if left unpollinated a female plant will divert all her energy to calyx production in an effort to seize pollen. This is especially desirable, as the calyx is where the highest concentration of trichomes exists, and the more densely packed a plant is with calyces, the greater psychoactive effect that plant will likely have. Potent sinsemilla is especially important to medical users, to minimize the amount of cannabis they must consume in order to be afforded relief.

Cannabis with seeds is generally considered to be of inferior quality and/or grown with inferior technique.

Indoors, plants like cannabis are induced into flowering by decreasing its photo period to at least 10 hours of darkness per day.

Traditionally plants lighting cycle to 12 hours on and 12 hours off. This change in photo period mimics the plant's natural outdoor cycle; with up to 18 hours of light per day in the summer and down to less than 12 hours of light come fall and winter.

While the flowering hormone in most plants (including cannabis) is present during all phases of growth, it is inhibited by exposure to light.
To induce flowering, the plant must be subject to at least 8 hours of darkness per day; this number is very strain-specific and most growers flower with 12 hours of darkness to be safe.
The flowering hormone is very quickly inhibited, taking less than two minutes of exposure.
Flowering usually lasts from 45 to 90 days indoors.
If growing outdoors it may take somewhat longer, depending on the natural onset of the colder seasons.

The flowering length is mainly genetically determined with plants (as pure cannabis “indica” strains) flowering in as low as 45 days, while plants (as cannabis “sativa”) can take up to 4 months to finish and the harvest yields significantly less. This is also the main reason why certain plants (as cannabis indica) are almost always grown indoors (unlike cannabis sativa, which is also grown outdoors).

In late flowering the calyx are easily visible to the naked eye. Calyx development begins approximately 1-2 weeks after the photo period is reduced. In the first weeks of flowering a plant usually doubles in size and can triple. Calyx development ends around 5 weeks into flowering and is proceeded by a period of Calyx “swelling”. During this time the buds greatly increase in weight and size.

### Outdoor Cannabis Cultivation

Cannabis can be planted outdoors under the sun, either on natural soil or in pots of pre-made or commercial soil. In most places of the subtropics cannabis is germinated from late spring to early summer and harvested from late summer to early autumn.

When cultivated outdoors, the chosen areas are those which receive twelve hours or more of sunlight in a given day. In the Northern Hemisphere cannabis seeds are typically planted in late May or early June, so the plants can have a full four months of growth. Typically, the plants are harvested anywhere from mid September to early October.

### Indoor Cannabis Cultivation.

Cultivating Cannabis indoors traditionally has to do with growing the plants in a soil-like medium and adding fertilizer when the plants are given water. Cultivating marijuana indoors is more complicated and expensive than growing outdoors, but it allows the cultivator complete control over the growing environment. Cannabis grown indoors can be just as potent as its outdoor counterpart if tended to properly.

Cultivating plants indoors can also be done through the use of hydroponics; however, this method is somewhat less common. In order to grow plants indoors, a growing medium (e.g. soil or growing substrate), water, nutrients, light and air need to be supplied to the plant.

### Supply of Light

To determine the appropriate lighting (and the best lamp to use), the specific needs of the plant must be considered, as well as the room size and ventilation. To arrange optimal lighting, the lighting present in the plant's natural environment needs to be imitated. For example vegetables grow best in full sunlight, which means in practice that as much light as possible must be supplied to grow cannabis indoors (high intensity discharge (HID) lights such as high pressure sodium (HPS) and metal halide (MH) are preferred. Fluorescent lamps can also be used). Incandescence and mercury vapor lighting are not used in cannabis cultivation.

In addition, plants also require both dark and light (“photo”-) periods.

As such, lights need to be timed to switch them on and off at set intervals. The optimum photo/dark-periods is specific depending on each plant (some prefer long days and short nights and others preferring the opposite, or something in between).

Most plants will grow under most light spectra, yet always prefer a full spectrum light (HPS).

However, certain plants (as cannabis) can be grown successfully under both types of light. MH is used for vegetative phase of growth, as it encourages short inter nodes (distance between sets of leaves), and inhibits cell elongation, creating a shorter, stockier plant. Metal halide lamps produce more ultraviolet radiation than high pressure sodium lamps, which may play a role in increasing the flowering (and for certain plants as cannabis the amount of working substances as THC) produced by the plant. High pressure sodium lamps trigger a greater flowering response in the plant and are thus used for the second (or reproductive) phase of the growth. If high pressure sodium lamps are used for the vegetative phase, plants will usually grow slightly more quickly, but will also have longer inter nodes, and may be taller.

Recent advancements in LED technology have allowed for diodes that emit enough energy for cannabis cultivation.

The HPS bulb has most of the light spectrum in the “orange” range, with almost no ‘blue’ and very little ‘red.’ For this reason, it is poor in the 430-460 nm, and poor in the 680-700 nm. Luckily, the light is so powerful that the spill-over at these frequencies is still sufficient to do a good job. The principal shortcoming of the HPS lamp turned it into an advantage for LEDs. LED lights allow one to focus intensity in the high PAR absorption range of the light spectrum. New models of LED grow lights incorporate multiple types of chips that cover the whole range of red light, blue light, and now full spectrum light.

One major short coming of LED's in the past has been a lack of intensity. Higher wattage chips are required to produce enough luminous efficiency to produce larger, denser yields. As with using a 400 w HPS vs. a 1000 w HPS, intensity has everything to do with yield. The same applies to LEDs however, it is not as simple as measuring watts because better quality chips can produce more light with less watts than cheap chips running at lower watts.

LED grow lights are still considered an experimental technology in cannabis cultivation. The market remains flooded with cheap quality LED lights that do not produce yields comparable to what growers are accustomed to. Many companies are using single watt LED chips, which have notoriously produced low yields and wispy results. Growers should look for lights with 6 watt chips. When considering purchasing LED grow lights, one should carefully examine both the spectrum and the intensity of the light.

The advantages of LEDs, low heat output, long life span, and simpler environmental control, coupled with the ever increasing quality of the technology ensure that they can potentially mark a significant transformation in the cultivation of cannabis. NASA has experimented with LED panel light sources on plant growth.

According to the inverse square law, the intensity of light radiating from a point source (in this case a bulb) is inversely proportional to the square of the distance from the source. So if an object is twice as far away, it receives only ¼ the light. This is a serious hurdle for indoor marijuana growers, and many techniques are employed to use light as efficiently as possible.

Reflectors are often used in the lamps to maximize light efficiency. Plants or lights are moved as close together as possible so that they receive equal lighting and that all light coming from the lamps wind up on the plants (rather than partly besides it). Often, the distance between lamp and plant is in the range of 0.6 m (2 ft) with incandescent lamps, to 10 cm (4 in) with other lamps, such as compact, large and high-output fluorescent lamps. Some marijuana cultivators cover the walls of their grow-room with some type of reflective material (often Mylar), or alternatively, white paint to maximize efficiency.

One commonly used covering is 6 millimeter (150 μm) PVC plastic sheeting that is white on one side and black on the other. The plastic is installed with the white side facing in to the room to reflect light, and the black facing the wall, to reduce fungus and mold growth. Another common covering is flat white paint, with a high titanium dioxide content to maximize reflectivity. Mylar sheeting from a grow store is very effective when it lines grow room walls, along with Astrofoil (which also reflects heat), and Foylon (a foil-laminated, reinforced fabric).

### Control of the Atmosphere

When growing indoors, the cultivator should maintain as close to an ideal atmosphere inside the grow-room as possible. The air temperature should be maintained within a specific range, typically with deviations no larger than 10° C. with a cooler night and warmer day. Adequate levels of CO2 must be maintained in order for the plants to grow most efficiently. It is also important to promote vigorous air circulation within the grow room, which is usually accomplished by mounting an extraction fan and one or more oscillating fans.

Assuming adequate light and nutrients are available to plants, the limiting factor in plant growth is the level of carbon dioxide (CO2). Plants grown with supplemental carbon dioxide will grow more quickly, have larger stomata, and can utilize more light. Ways of increasing carbon dioxide levels in the grow-room include: bottled carbon dioxide, carbon dioxide generators, a milk jug and yeast solution (in which yeast grows in a container thereby emitting CO2), a baking soda and vinegar mixture in a container, or dry ice.

### Harvesting, Drying and Curing

Close-up examination of a female marijuana bud in flowering stage displays white trichomes seen coating the surface, which will darken as flowering progresses.

A typical indicator that a plant is ready to be harvested is when ⅔ of the pistils have turned from white to reddish brown or other color. In general, harvesting consists of drying and curing. Curing is essential for the even distribution of moisture in the buds. A popular alternate method is the following:

Dry: Buds left in well ventilated dark place for 24 hours

Cure: Buds stored in sealed bag and left in dark place for 8 hours

Dry: Buds left in well ventilated dark place for 16 hours

Cure: Buds stored in sealed bag and left in dark place for 6 hours

Dry: Buds left in well ventilated dark place for 12 hours

Steps continued likewise as necessary In 3-4 days buds are ready for consumption.

Cannabis buds are typically harvested when fully ripe. Generally, ripeness is defined as when the white pistils start to turn dark yellow, orange, light to mid red, etc. and the trichomes, “crystals”, barely begin to turn milky from clear. These trichomes can range from completely clear (generally deemed underdeveloped), to amberish-red. Ideally, professionals will use a decent power magnifying glass, a brix meter (to measure “sugar” content), and a microscope. The potential seed pods swell with resins usually reserved for seed production, thus improving the quality of the buds (called colitas, Spanish for “little tails”), which will swell to form full “colas” (Spanish for “tails”). If harvested early on with only a few of the pistils turned color, the buds will have a more pure THC content and less of the cannabinoids CBD and CBN. The latter cannabinoids are non-psychoactive; they contribute to the bouquet of the marijuana and modulate the overall nature of the high anywhere from purely psychedelic to purely sedative.

Contrary to sinsemilla (bud production focused cultivation), seeds are harvested when fully developed and often after the accompanying buds have begun to deteriorate.

### Drying

The plants are dried at room temperature in a dark space. This process can take from a few days to two weeks, depending on the size and density of the buds and the relative humidity of the air. A stable temperature preserves cannabinoids well. Some believe flowers are hung by their stalks, allowing the internal fluids of the plant to remain in the flowers. Others believe the cut stem is simply a handy non-sticky place from which to hang the plant. Roots are removed. When the stems in the middle of the largest buds can be snapped easily, the plant is dry enough to be cured. Drying is done in a dark place, as THC resins will deteriorate if exposed to light and the degradation product CBN will be formed, thus significantly altering the cannabinoid profile of the dried flowers.

### Curing

The curing process continues breaking down sugars and helps develop taste and smoothness of smoke. Usually, the dried product is packed (not compressed) into glass canning jars which are airtight. Initially the product is checked periodically (every few hours) to make sure it was properly dried and has not re-moistened itself. After several days, when the product is dried to satisfaction, the jars are sealed off and opened just once a week.

Curing is highly varied—the minimum is usually two weeks. Some growers even cure as long as six months, while others do not cure at all. As with tobacco, curing can make the cannabis more pleasant to smoke. For the same reasons as when drying, curing jars are stored in a cool, dark place.

A recent method of curing is called water curing. This method is quicker and can improve a lower quality product. The freshly cut buds are submersed in water for a period of 7 straight days, changing the water daily. The buds are then dried and are ready to use. Nutrients can be added to the plants up until they are harvested. When water curing, the water will flush out harmful chemicals (such as the ones used to feed the plants) as well as proteins, sugars, pigments, chlorophyll and some resins. This will also increase the THC to weight ratio. Many believe the finished product is not as attractive as using a standard dry and cure

### Pests

Outdoor growers are likely to confront issues regarding pests. In any case (indoor or outdoor), experienced growers recommend caution when using chemical pesticides, for they may have toxic effects on the environment, the plants themselves and in turn cannabis consumers. As a general rule, experts mandate the deployment of pesticides clearly marked as “safe to use on food crops”. Substances proven to induce little or no harm include:

Pyrethrins: Organic and very effective, although sometimes hard to find. Often expensive due to high production cost.

Azadirachtin: Meets most criteria to be classified as natural insecticide. Biodegradable, non-toxic to mammals. Usually cheaper and easier to find than pyrethrins.

Indoor growers also have problems with pests, if caught too late, eradication of many destructive insect species indoors may be impossible until all infected plants are removed from the space and sterilization methods employed.

### Advanced Cultivation Methods

### Hydroponics

An example for a small hydroponic system for cannabis cultivation Hydroponic cultivation generally occurs indoors, although there is no practical obstacle to growing outdoors. In general, it consists of a non-soil medium which is exposed to a nutrient and water flow.

There are many types of hydroponic systems. If the nutrient solution floods the loose growing medium and recedes for aeration, this is an ebb and flow or flood and drain system. Systems that gradually drip solution onto the medium are drip systems. Systems that intermittently spray roots floating in air are called aeroponic systems. If aerated water runs down a channel lined with a film of rooting medium, this is a nutrient film technique system. A series of tubes intermittently running high flow nutrient solution into the tops of growing containers use a top feed system.

Hydroponic systems greatly increase aeration of plant roots, and increase control of nutrient uptake. Hydroponic systems are decidedly more difficult to operate for the amateur or hobby grower, as over-fertilization is common, because there is no soil to act as a nutrient buffer. For this reason, many growers now use coconut fiber as a soil-less medium due to its high drainage and buffering capabilities, making it almost impossible to over-fertilize. Additionally, if a hydroponic system fails, the crop has a high probability of dying as the roots rapidly dry out (this is especially true of aeroponic systems).

There is now a new breed of hydroponic configurations such as the Omega Garden, the B-Pod and the EcoSystem Vertical Growing System that use circular designs to maximize efficiency. This consists of plants being placed or, in the case of the Omega Garden, revolving around a central light which makes maximum use of the light output.

### Genetics and Breeding

Selection of Mother Plants

An important factor in cannabis cultivation is selecting the best genetics for one's crop. This is frequently done by selecting one or more known strains, or strains with preferred genetics (in the case of marijuana, one might use seeds from a batch that was particularly engineered), and then growing a number of the seeds to find out which exhibit the characteristics most desirable to the cultivator. These genetics should typically yield the fluorescence of interest.

Plant characteristics which are generally selected for include:

Overall yield

Time to fruition

Resistance to pests

Geometric traits (uniformity, compactness, flower density, fluorescence etc)

Color

Flavor and/or aroma

Appeal to end buyer (known as “bag appeal”)

Psychoactive qualities

Trichome density and type (stalked or sessile)

When a cultivator has decided which plant or plants exhibit the most desirable traits, a cutting is taken and grown to maturity but never allowed to flower. This plant is referred to as a mother, and can be kept for years, producing thousands of clones genetically identical to the mother.

### Hybrid Vigor

When crossing two strains of cannabis (or two of any plant), the resultant hybrid may possess what is called hybrid vigor. In general, this produces a plant which is healthier, stronger, or quicker growing than its predecessors.

Sometimes, in the case of a plant which has been brought back from fruiting (fruition, as mentioned above), it may be beneficial to cross it back with another (close) relative, in the hopes that it will become invigorated. Caution should be exercised, as one does not always attain a beneficial cross with hybridizing.

Cloning from Cuttings

Like many plants, cloning of cannabis is possible through a relatively simple process. The process itself is quite similar to the cloning of most other plants and involves rooting branch cuttings from donor (“mother”) plants.

There are many methods of cloning available, from store bought purpose built cloning machines to inserting a cutting in a cup of water and waiting for roots to grow. Most methods will take anywhere from 5-21 days.

Rooting hormone gels or powder mixes are applied to the cut to promote root growth and inhibit fungal infection. The cutting is then placed in a rooting medium which may be a soil mix or a soil-less medium. Typical soil-less media are Perlite, vermiculite, peat moss, sand, rock wool or Oasis foam. A good medium is one that drains well, holds moisture and air well also. Oxygen is important for healthy root growth.

The cuttings in their new medium should be kept at a constant temperature (around 78 F) and with high humidity. Elevated humidity levels can be achieved by use of a humidifier or a humidity dome. Elevated humidity levels slow the transpiration rate which is important because without a root system the water uptake is very slow; If the transpiration rate exceeds the uptake rate the cutting is losing water and will wilt and die.

Many growers use a humidity dome as they are very inexpensive, around $7, and are easy to use. Many others improvise domes with simple plastic baggies secured with rubber bands (even less expensive and equally easy to use). When using a humidity dome, the dome should be removed at least twice a day and the rooting clones should be fanned to prevent mold and to give them some air circulation. Alternatively, you can cut off the bottom of a clear 3-liter bottle and temporarily put it over a single plant. The cap can easily be removed a couple times a day to easily re-freshen air.

New Clones Under Improvised Humidity Domes.

The rooting medium should be kept moist and should never dry out. During other stages of growth one is advised to allow the soil to dry out to allow the roots to get oxygen and to prevent root rot. Since cuttings do not have roots this is not of concern. What is of concern is that a cutting will dry out and die, which occurs very rapidly.

Light intensity should be very low during the rooting process. High light intensities will force the plant to focus on photosynthesis at the expense of rooting. Light intensity should be increased during the last week up to normal illumination levels.

Cuttings usually take 7-14 days to develop root systems. Drooping is common within the first week. Cuttings that have not regained rigidity after 7 days are weak and are culled by most growers. To speed the rooting process keep the cuttings at constant temperature. Allowing the parent plant to become mildly nitrogen deficient before the cutting is taken will also speed rooting.

If performed correctly, the cuttings should stay green during their rooting time, and condensation should appear on the plastic coverings for the cuttings, which indicates proper humidity. After 7 days, healthy cuttings will appear strong with leaves reaching upward. Yellowing leaf tips are a common indicator of successful rooting. Browning likely indicates too much sunlight, too little humidity, cutting rotting in sitting water, or unsanitary cloning conditions.

In recent years, stores selling hydroponic grow equipment began offering automated machines (i.e.: EZCIoner, etc ˜$300 USD.) in which trimmed cuttings are placed and left alone for approximately two weeks. Established growers indicate these automated machines have near 100% success rates.

### Topping

Is done by removing the top of the apical meristem (dominant central stem), called the apex or terminal bud, in order to transfer apical dominance (the tendency for the apex to grow more rapidly than the rest of the plant) to the shoots emanating from the two nodes immediately beneath the pruning cut. This process can be repeated on one or both of the two new meristems, when they become apically dominant, with the same results. This process can actually be repeated almost infinitely, but over-diffusion of apical dominance will produce smaller, lower quality buds, so it is usually done no more than a few times. Topping also causes more rapid growth of all of the branches below the cut while the plant heals.

### Pinching

Pinching (also called super cropping) is similar to topping in that it causes the lower branches to grow more rapidly, but the apical meristem will maintain apical dominance, which is especially useful if the plant has already been topped. Pinching is performed by firmly pinching the apical meristem(s) so as to substantially damage vascular and structural cells but without totally breaking the stem. This will cause the lower limbs to grow more rapidly while the pinched tissue heals, after which time the stem will resume apical dominance.

### LSTing

LST stands for Low Stress Training and is another form of supercropping, many times referred to as LST super cropping. This technique involves bending and tying the plants branches to manipulate the plant into a more preferred growth shape. This method of training works very well for indoor growers who need to illuminate their plants using overhead lights. Since light intensity greatly diminishes with increased distance (Inverse—square law) LSTing can be used to keep all growth tips (meristem) at the same distance from the light and can achieve optimal light exposure. LST can be used in conjunction with topping, since topping increases axial growth (side shoots), topping is often done a few weeks before beginning LSTing. LSTing works by changing the distribution of hormones, more specifically Auxins, in the plant.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While the foregoing is directed to embodiments of the present invention, other and further embodiments of the invention may be devised without departing from the basic scope thereof, and the scope thereof is determined by the claims that follow. Any claim below that is written as dependent on an independent claim can also be written as dependent on any of the claims under such independent claim, except where logic forecloses such a dependency.

### Numbered Embodiments of the Invention

1. Biologically, physically, or chemically transform Cannabis species to express genetic and/or fluorescent markers for detection of Medical Marijuana from illicit Marijuana sources.
2. A Cannabis plant, or a part thereof, wherein a sample of plant material can be made readily detectable by authorities using genetic and/or fluorescent markers.
3. A transformed Cannabis plant that can identify medical marijuana from other forms of marijuana for production, distribution, and sale.
4. A transformed Cannabis plant that can be identified with detection devices for genetic and fluorescent markers.
5. A transformed Cannabis plant that can be identified with fluorescent and/or genetic markers built into the expression vectors or plasmids or within separate vectors or plasmids.
6. The Cannabis plant part of embodiment 1, wherein said plant part is regenerable.
7. The Cannabis plant part of embodiment 1, wherein said plant part is a constituent part of the plant, plant product, plant flower, a clone or a single cell.
8. A tissue culture of regenerable cells of a transformed Cannabis plant or a part thereof.
9. The tissue culture of embodiment 8, wherein the regenerable cells are from embryos, meristematic cells, pollen, leaves, roots, root tips, anther, pistil, flower, seed, bud, or stem.
10. A Cannabis plant regenerated from the tissue culture of embodiment 8, wherein the regenerated Cannabis plant expresses all of the physiological and morphological characteristics of transformed Cannabis.
11. A method of producing Cannabis seed, comprising crossing the plant of embodiment 1 with itself or a second Cannabis plant.
12. The method of embodiment 8, wherein said method comprises crossing the plant of embodiment 8 with a second, distinct Cannabis plant.
13. An F.sub.1 hybrid Cannabis seed produced by the method of embodiment 9.
14. An F.sub.1 hybrid Cannabis plant produced by growing the seed of embodiment 10.
15. A method of producing a Cannabis plant having an added desired trait, wherein the method comprises introducing a transgene conferring the desired trait into the Cannabis plant of embodiment 1.
16. The method of embodiment 12, wherein the desired trait confers detectable characteristics to the Cannabis plant, constituent parts, seeds, buds, trichomes, leaves, stems, roots etc.
17. The method of embodiment 13, wherein the desired trait confers a genetic marker and/or a fluorescent marker to a subsequent generations.
18. The method of embodiment 12, wherein the desired trait is a detectable marker and the transgene encodes a fluorescent protein and/or genetic marker sequence.
19. A Cannabis plant produced by the method of embodiment 12, wherein the plant comprises the desired trait and all of the physiological and morphological characteristics of Cannabis when grown in the same environmental conditions.
20. A method of introducing a detectable marker into Cannabis comprising: (a) crossing a transformed Cannabis plant, with a second plant comprising a detectable marker to produce F1 progeny plants; (b) selecting F1 progeny plants that have the detectable marker to produce selected F1 progeny plants; (c) crossing the selected progeny plants with at least a first transformed Cannabis plant to produce backcross progeny plants; (d) selecting backcross progeny plants that have the detectable marker and physiological and morphological characteristics of transformed Cannabis to produce selected backcross progeny plants; and (e) repeating steps (c) and (d) one or more times in succession to produce selected second or higher backcross progeny plants that comprise the detectable marker and otherwise comprise all of the physiological and morphological characteristics of Cannabis when grown in the same environmental conditions.
21. The method of embodiments 1, 8, 12, 13, 14, 17, 18 wherein the detectable marker confers a trait, wherein the trait is at least one of detectability, or combinations thereof.
22. The method of embodiments 1, 8, 12, 13, 14, 17, 18, wherein the trait is detectable and the detectability is conferred by Agrobaterium containing an engineered Ti plasmid containing a transgene encoding a fluorescent protein and a genetic marker sequence either within the plasmid with the fluorescent protein or within a separate vector/plasmid.
23. The method of embodiments 1, 8, 12, 13, 14, 17, 18, wherein the trait is detectable and the detectability is conferred by microinjection containing an engineered Ti plasmid containing a transgene encoding a fluorescent protein and a genetic marker sequence either within the plasmid with the fluorescent protein or within a separate vector/plasmid.
24. The method of embodiments 1, 8, 12, 13, 14, 17, 18, wherein the trait is detectable and the detectability is conferred by chemical/biochemical technique (Polyethanol Glycol PEG, Dextran transfection, Calcium phosphate transfection etc) containing an engineered Ti plasmid containing a transgene encoding a fluorescent protein and a genetic marker sequence either within the plasmid with the fluorescent protein or within a separate vector/plasmid.
25. The method of embodiments 1, 8, 12, 13, 14, 17, 18, wherein the trait is detectable and the detectability is conferred by gene/particle bombardment using Gold or other particle microcarriers to coat an engineered Ti plasmid containing a transgene encoding a fluorescent protein and/or a genetic marker sequence either within the plasmid with the fluorescent protein or within a separate vector/plasmid for delivery into the host Cannabis tissue or cells.
26. A Cannabis plant produced by the method of embodiments 1, 8, 12, 13, 14, 17, 18, wherein the transformed Cannabis plant has the desired detectable marker(s) and all of the physiological and morphological characteristics of Cannabis.
27. A method of producing an inbred Cannabis plant derived from the transformed Cannabis, the method comprising the steps of: (a) preparing a progeny plant derived from transformed Cannabis by crossing transformed Cannabis with a Cannabis plant of a second variety; (b) crossing the progeny plant (transformed Cannabis) with itself or a second plant to produce a seed of a progeny plant of a subsequent generation; (c) growing a progeny plant of a subsequent generation from said seed and crossing the progeny plant of a subsequent generation with itself or a second plant; and (d) repeating steps (b) and (c) for an additional 3-10 generations with sufficient inbreeding to produce an inbred Cannabis plant derived from the transformed Cannabis.
28. A method of producing a commodity plant product comprising obtaining the plant or plant part of embodiment 1 and producing said commodity plant product therefrom.
29. The method of embodiment 23, wherein the commodity plant product transformed Cannabis and all constituent components of the Cannabis.
30. A transformed Cannabis plant produced by growing the seed of embodiment 1.
31. A protoplast produced from the tissue culture of embodiment 5.
32. A method of producing a transformed Cannabis plant having an added desired trait comprising introducing a transgene conferring the desired trait into the plant of embodiment 7.
33. A method of producing a transformed Cannabis plant having an added desired trait comprising introducing a transgene conferring the desired trait into the plant of embodiment 25.
34. A transformed Cannabis plant produced by the method of embodiment 27, wherein the plant comprises the desired trait and all of the physiological and morphological characteristics of Cannabis when grown in the same environmental conditions.
35. A transformed Cannabis plant produced by the method of embodiment 28, wherein the plant comprises the desired trait and all of the physiological and morphological characteristics of Cannabis when grown in the same environmental conditions.
36. A Cannabis plant can be transformed to express fluorescent protein(s) in a vector or plasmid that expresses the fluorescent protein(s) of interest in all components of the Cannabis plant (ie. Stems, cells, tissue, seeds, flowers, buds etc.)
37. Any color from the UV, visible, near infra-red, and far infra-red spectrums can be expressed in a plasmid or vector to create a self-replicating, transformed Cannabis species.
38. Transformed Cannabis expressing fluorescent proteins/markers can be used to detect medical Cannabis “marijuana” from illegal/common Cannabis “marijuana” species or plants and plant material as stated in embodiment 8, 9, and 36.
39. IA genetic sequence can be expressed within an expression vector expressing a fluorescent protein of my choice from embodiment 37 to add an additional level of detection of medical Cannabis “marijuana” from illegal/common Cannabis marijuana species or plants and plant material as stated in embodiment 8, 9, and 36.
40. A genetic sequence can be expressed or co-expressed from embodiment 39 within a separate vector or plasmid as an additional level of detection of medical Cannabis “marijuana” from illegal/common Cannabis “marijuana” species or plants and plant material as stated in embodiment 8, 9, and 36.
41. A method of intrinsically marking a Cannabis plant comprising stably transforming the plant to express an extrinsic bio-marker.
42. The method of embodiment 41, wherein the biomarker is a fluorescent bio-marker and the further comprising stably transforming the plant to express a second extrinsic fluorescent bio-marker, distinguishable from the first by excitation or emission wavelength.
43. A method of intrinsically marking a Cannabis plant comprising stably transforming the plant to express a stably incorporated extrinsic segment of coding-marker, the coding-marker readable for particular information on the source of the Cannabis plant.
44. A Cannabis plant stably transformed to express a stably incorporated extrinsic segment of coding-marker, the coding-marker readable for particular information on the source of the Cannabis plant.

### CITED REFERENCES

### Agrobacterium Tumerfaciens

Chilton M D, Drummond M H, Merio D J, Sciaky D, Montoya A L, Gordon M P, Nester E W, Stable incorporation of plasmid DNA into higher plant cells: the molecular basis of crown gall tumorigenesis, Cell. 1977 June; 11(2):263-71.
Moore L W, Chilton W S, Canfield M L. 1997. Diversity of Opines and Opine-Catabolizing Bacteria Isolated from Naturally Occurring Crown Gall Tumors. App. Environ. Microbiol. 63:201-207.
Stanton B. Gelvin, Department of Biological Sciences, Purdue University, West Lafayette, Ind. 47907-1392, Agrobacterium-Mediated Plant Transformation: the Biology behind the “Gene-Jockeying” Tool, http://mmbr.asm.org/cgi/reprint/67/1/16
Stanton B. Gelvin, Department of Biological Sciences, Purdue University, West Lafayette, Ind. 47907-1392, Agrobacterium-Mediated Plant Transformation: the Biology behind the “Gene-Jockeying” Tool, http://mmbr.asm.org/cgi/reprint/67/1/16
Zupan J, Muth T R, Draper O, Zambryski P. 2000. The transfer of DNA from Agrobacterium tumefaciens into plants: a feast of fundamental insights. Plant J. 23:11-28.
Goodner B, Hinkle G, Gattung S, Miller N, et al. 2001. Genome Sequence of the Plant Pathogen and Biotechnology Agent Agrobacterium tumefaciens C58. Science. 294:2323-2328.
Wood D W, Setubal J C, Kaul R, Monks D E, et al. 2001. The Genome of the Natural Genetic Engineer Agrobacterium tumefaciens C58. Science. 294:2317-2323.
Vaudequin-Dransart V, Petit A, Chilton W S, Dessaux Y. 1998. The cryptic plasmid of Agrobacterium tumefaciens cointegrates with the Ti plasmid and cooperates for opine degradation. Molec. Plant-microbe Interact. 11:583-591.
Schell J, Van Montagu M., The Ti-plasmid of Agrobacterium tumefaciens, a natural vector for the introduction of nif genes in plants?, Basic Life Sci. 1977; 9:159-79.
Zambryski P. et al. 1983. Ti plasmid vector for introduction of DNA into plant cells without alteration of their normal regeneration capacity. EMBO J. 2:2143-2150.
Root M. 1988. Glow in the dark biotechnology. Bioscience. 38:745-747.
Kunik T, Tzfira T, Kapulnik Y, Gafni Y, Dingwall C, Citovsky V. 2001. Genetic transformation of HeLa cells by Agrobacterium. Proc. Natl. Acad. Sci. 98:1871-1876.
Dickinson, M. (2003). Molecular Plant Pathology. BIOS Scientific Publishers. Lal, Erh-Min and Kado, Clarence I. (2000). The T-Pilus of Agrobacterium tumefaciens. Trends in Microbiology, Vol. 8, Issue 8.
Ward, Doyle V., Zupan, John R and Zambryski, Patricia C. (2002). Agrobacterium VirE2 gtes the VIP1 treatment in plant nuclear import. Trends in Plant Science, Vol. 7 Issue 1.

### Lipofection Transfections, Chemical Transformations

Feigner P L et al. (1987) Lipofection: a highly efficient, lipid-mediated DNA-transfection procedure. Proc Natl Acad Sci USA 84: 7413-7417.
Feigner J H et al. (1994) Enhanced gene delivery and mechanism studies with a novel series of cationic lipid formulations. J Biol. Chem. January 28; 269(4):2550-61.

1. A Cannabis plant stably transformed to express an extrinsic bio-marker.

2. The plant of claim 1, wherein the biomarker is a fluorescent bio-marker.

3. The plant of claim 2, stably transformed to express a second extrinsic fluorescent bio-marker, distinguishable from the first by excitation or emission wavelength.

4. The plant of claim 1, stably transformed to express a second extrinsic bio-marker, distinguishable from the first.

5. The plant of claim 1, wherein the biomarker is detectable after contacting plant tissue or homogenate with an extrinsic substrate.

6. The plant of claim 1, further comprising a stably incorporated extrinsic segment of coding-marker, the coding-marker readable for particular information on the source of the Cannabis plant.

7. A method of distinguishing medical Cannabis from illicit Cannabis comprising:
if needed contacting a sample of the plant with a suitable substrate; and
detecting for the presence of a light-based indicator not present in wild-type Cannabis.

8. The method of claim 7, wherein a suitable substrate is contacted with the plant sample prior to the detecting.

9. The method of claim 7, wherein detecting is for the presence of a fluorescent extrinsic bio-marker.

10. The method of claim 9, further comprising detecting for the presence of a second extrinsic fluorescent bio-marker, distinguishable from the first by excitation or emission wavelength.

11. The method of claim 7, further comprising detecting for the presence of a second extrinsic bio-marker, distinguishable from the first.

12. The method of claim 11, outputting from a controller that derived or received the detecting results a report on containing for particular information on the source of the Cannabis plant derived from detecting for the presence of two or more extrinsic bio-markers.

13. The method of claim 7, further comprising detecting for the presence of a coding-marker.

14. The method of claim 7, further comprising measuring the concentration of THC in the plant and comparing that concentration to that appropriate for a plant with its bio-marker(s).

15. The method of claim 7, wherein the detecting is for the presence of a light-based indicator not present in wild-type Cannabis is a integrally mixed sub-part of the plant sample.

16. The method of claim 15, wherein the detecting is for the presence of a light-based indicator not present in wild-type Cannabis is of two or more distinct integrally mixed sub-parts of the plant sample.

17. A mobile detector of licit Cannabis from prospective Cannabis comprising:
a controller;
an optical detector operative to send optical data derived from a sample of the Cannabis to the controller; and
one or more output devices through which the controller delivers information on whether the Cannabis is licit or illicit based on the optical data, wherein the controller includes programming for deriving a report on whether the Cannabis is licit or illicit based on the optical data.

18. The detector of claim 17, wherein the optical detector is a fluorescence detector.

19. The detector of claim 17, wherein ratios to two or more fluorescent signals are used to determine if the Cannabis is licit or illicit.

20. The detector of claim 17, further comprising: a biometrics detector operatively connected to the controller.

21. The detector of claim 20, wherein the output device(s) include a printer adapted with the controller to print sample container labels based on information, accessed by the controller, on the holder of the prospective Cannabis.
